(19) United States
(12) Patent Application Publication
(10) Pub. No.: US 2024/0327506 A1
(43) Pub. Date: Oct. 3, 2024

(54) METHODS AND COMPOSITION FOR THE SELECTIVE ENHANCEMENT OF ADOPTIVE CELL THERAPY

(71) Applicant: MaineHealth, Portland, ME (US)

(72) Inventors: Xianghua Han, Scarborough, ME (US); Jennifer M. Caron, Scarborough, ME (US); Peter C. Brooks, Harpswell, ME (US)

(21) Appl. No.: 18/620,778

(22) Filed: Mar. 28, 2024

Related U.S. Application Data
(60) Provisional application No. 63/455,471, filed on Mar. 29, 2023.

(51) Int. Cl. C07K 16/18; A61K 35/17; A61P 35/00; C07K 16/28

(52) U.S. Cl. CPC C07K 16/18; A61K 35/17; A61P 35/00; C07K 16/2818; C07K 16/2827; C07K 16/2848

(57) ABSTRACT

The present invention provides compositions and methods of use thereof in the treatment of cancer and abnormal immune suppression diseases by enhancing adoptive cell therapy. In some embodiments, the methods provided herein increase T-cell infiltration into a tumor.

### RELATED APPLICATION

The instant application claims priority to U.S. Provisional Application No. 63/455,471, filed Mar. 29, 2023, the entire contents of which are expressly incorporated by reference herein.

### GOVERNMENT SUPPORT

This invention was made with government support under grant numbers CA196739, awarded by the National Institutes of Health/National Cancer Institute. The government has certain rights in the invention.

### SEQUENCE LISTING

The application contains a Sequence Listing which has been submitted electronically in .XML format and is hereby incorporated by reference in its entirety. Said .XML copy, created on Mar. 28, 2024, is named “136191-00402.xml” and is 20,978 bytes in size. The sequence listing contained in this .XML file is part of the specification and is hereby incorporated by reference herein in its entirety.

### BACKGROUND OF THE INVENTION

Adoptive cell therapy (ACT) is an immune cellular therapy that involves using isolated tumor-infiltrating T-cells (TILs) or genetically engineered T-cells that are infused into a subject to treat diseases such as cancer (1-3). ACT is based in part on the idea that specific subsets of effector T-cells directed to tumor antigens can be expanded ex-vivo and then re-infused into patients in hopes that these cells will rapidly localize to the malignant lesion and efficiently kill the malignant tumor mass. A central caveat of this therapeutic approach is that the T-cells have an efficient mechanism that allows the cells that have been injected into the circulation, to migrate out of tumor associated blood vessels in order to gain access to the tumor (1-3). As such, prior to the invention described herein, there was a pressing need to develop methods and compositions to improve ACT access to tumor tissues and thereby improve the effectiveness of ACT therapeutics.

### SUMMARY OF THE INVENTION

Accordingly, in one aspect, the present invention provides a method of treating cancer in a subject, the method comprising administering to the subject an adoptive cell therapeutic composition; and an antagonist of collagen or a functional fragment thereof, thereby treating cancer in the subject.

In another aspect, the present invention provides a method of increasing the efficacy of adoptive cell therapy or T-cell therapy in a subject having cancer, the method comprising administering to the subject an adoptive cell therapeutic composition; and an antagonist of collagen or a functional fragment thereof, thereby increasing the efficacy of the adoptive cell therapy or T-cell therapy compared to a control.

In another aspect, the present invention provides a method of modulating T-cell infiltration into a tumor in a subject, the method comprising administering to the subject an adoptive cell therapeutic composition; and an antagonist of collagen or a functional fragment thereof, thereby modulating T-cell infiltration into the tumor in the subject compared to a control.

In another aspect, the present invention provides a method of modulating Treg and/or myeloid-derived suppressor cell (MDSC) levels in a tumor in a subject, the method comprising administering to the subject an adoptive cell therapeutic composition; and an antagonist of collagen or a functional fragment thereof, thereby modulating Treg and/or myeloid-derived suppressor cells (MDSCs) levels in the tumor in the subject compared to a control.

In one embodiment, the T-cell infiltration into the tumor of the subject is increased compared to the control. In one embodiment, the Treg and/or MDSC levels are decreased in the tumor in the subject compared to the control. In one embodiment, the control is prior to administration of the adoptive cell therapeutic composition, and the antagonist of collagen or the functional fragment thereof.

In one embodiment, the adoptive cell therapeutic composition comprises a cell type selected from a group consisting of a tumor infiltrating lymphocytes (TIL), T-cell receptor modified lymphocytes and chimeric antigen receptor modified lymphocytes. In one embodiment, the adoptive cell therapeutic composition comprises tumor infiltrating lymphocytes (TIL). In one embodiment, the adoptive cell therapeutic composition comprises a cell type selected from a group consisting of T-cells, CD8+ cells, CD4+ cells, NK-cells, delta-gamma T-cells, regulatory T-cells, and peripheral blood mononuclear cells. In one embodiment, the adoptive cell therapeutic composition comprises T-cells.

In one embodiment, the collagen comprises collagen type-I, collagen type II, collagen type III, or collagen type-IV. In one embodiment, the antagonist of collagen and functional fragment thereof comprises an antagonist of collagen type-I or a functional fragment thereof, or an antagonist of collagen type-IV or a functional fragment thereof. In one embodiment, the antagonist of collagen type-I or the functional fragment thereof comprises an antagonist of the XL313 cryptic collagen epitope, or an antagonist of the HU177 cryptic collagen epitope. In one embodiment, the antagonist of collagen type-I or the functional fragment thereof comprises an antibody or an antigen-binding fragment thereof that binds a cryptic RGDKGE (SEQ ID NO: 1) containing collagen epitope. In one embodiment, the antagonist of collagen type-I or the functional fragment thereof comprises an antibody or an antigen-binding fragment thereof that binds a cryptic CPGFPGFC (SEQ ID NO: 16) containing collagen epitope. In one embodiment, the antibody or the antigen-binding fragment thereof comprises a monoclonal antibody or an antigen-binding fragment thereof. In one embodiment, the monoclonal antibody or antigen-binding fragment thereof comprises an XL313 monoclonal antibody or an antigen-binding fragment thereof. In one embodiment, the monoclonal antibody or antigen-binding fragment thereof comprises an HU177 monoclonal antibody or an antigen-binding fragment thereof.

In one embodiment, administration of the antagonist of collagen or the functional fragment thereof increases CD8+ cells in a tumor in the subject by at least 1.1-fold, at least 1.2-fold, at least 1.5-fold, at least 2-fold, at least 3-fold, at least 4-fold, at least 5-fold, at least 10-fold, compared to a control. In one embodiment, administration of the antagonist of collagen or the functional fragment thereof increases CD8+ cells in ascites fluid in the subject by at least 1.1-fold, at least 1.2-fold, at least 1.5-fold, at least 2-fold, at least 3-fold, at least 4-fold, at least 5-fold, at least 10-fold, compared to a control. In one embodiment, administration of the antagonist of collagen or the functional fragment thereof decreases CD4+ Treg cells in a tumor in the subject by at least 1.1-fold, at least 1.2-fold, at least 1.5-fold, at least 2-fold, at least 3-fold, at least 4-fold, at least 5-fold, at least 10-fold, compared to a control. In one embodiment, administration of the antagonist of collagen or the functional fragment thereof decreases CD4+ Treg cells in ascites fluid in the subject by at least 1.1-fold, at least 1.2-fold, at least 1.5-fold, at least 2-fold, at least 3-fold, at least 4-fold, at least 5-fold, at least 10-fold, compared to a control. In one embodiment, administration of the antagonist of collagen or the antigen-binding fragment thereof decreases MDSC cells in a tumor in the subject by at least 1.1-fold, at least 1.2-fold, at least 1.5-fold, at least 2-fold, at least 3-fold, at least 4-fold, at least 5-fold, at least 10-fold, compared to a control.

In one embodiment, the migration of T-cells to the tumor in the subject is increased compared to a control. In one embodiment, the migration of CD8+ T-cells to the tumor in the subject is increased compared to a control. In one embodiment, the migration of CD4+ Treg-cells and/or MDSCs to the tumor in the subject is decreased compared to a control.

In one embodiment, the level of phosphorylation and/or activation of a P38 MAP Kinase in the subject is inhibited compared to a control.

In one embodiment, the control is prior to administration of the adoptive cell therapeutic composition, and the antagonist of collagen or the antigen-binding fragment thereof.

In one embodiment, the methods as provided herein further comprise administering an antagonist of an integrin to the subject. In one embodiment, the integrin comprises integrin αvβ3. In one embodiment, the antagonist of integrin αvβ3 comprises an antibody capable of binding an RGDKGE (SEQ ID NO: 1) containing collagen epitope.

In one embodiment, the adoptive cell therapeutic composition and the antagonist of collagen or the functional fragment thereof are administered simultaneously to the subject. In one embodiment, the adoptive cell therapeutic composition and the antagonist of collagen or the functional fragment thereof are administered consecutively, in any order, to the subject. In one embodiment, the adoptive cell therapeutic composition, the antagonist of collagen or the functional fragment thereof, and the antagonist of an integrin are administered simultaneously to the subject. In one embodiment, the adoptive cell therapeutic composition, the antagonist of collagen or the functional fragment thereof, and the antagonist of an integrin to the subject is conducted consecutively, in any order.

In one embodiment, there is a time period of one minute to four weeks between the consecutive administration of the adoptive cell therapeutic composition and the antagonist of collagen or the functional fragment thereof and/or there are several administrations of the adoptive cell therapeutic composition and the antagonist of collagen or the functional fragment thereof. In one embodiment, the administration of the adoptive cell therapeutic composition and/or the antagonist of collagen or the functional fragment thereof is conducted through an intra-tumoral, intra-arterial, intravenous, intrapleural, intravesicular, intracavitary or peritoneal injection, or an oral administration.

In one embodiment, the methods provided herein further comprise administering concurrent or sequential radiotherapy, monoclonal antibodies, chemotherapy, immunotherapy or other anticancer drugs or interventions to the subject. In one embodiment, the immunotherapy comprises administration of an immune checkpoint inhibitor to the subject. In one embodiment, the immune checkpoint inhibitor comprises an inhibitor of CTLA-4, PD-1, PDL-1, Lag3, LAIR1, or LAIR 2. In one embodiment, the immune checkpoint inhibitor comprises an anti-CTLA-4 antibody, an anti-PD-1 antibody, an anti-PDL-1 antibody, an anti-Lag3 antibody, an anti-LAIR1 antibody, or an anti-LAIR 2 antibody. In one embodiment, the immune checkpoint inhibitor comprises an anti-PD-1 antibody, or an anti-PDL-1 antibody.

In one embodiment, the subject is a human.

In one embodiment, the cancer is selected from the group comprising of melanoma, central nervous system (CNS) cancer, CNS germ cell tumor, lung cancer, leukemia, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, or hematological cancer. In one embodiment, the hematological cancer is a leukemia or a lymphoma, optionally wherein the leukemia is acute lymphoblastic leukemia (ALL), acute myelogenous leukemia (AML), chronic lymphocytic leukemia (CLL), small lymphocytic lymphoma (SLL), chronic myelogenous leukemia (CML) or acute monocytic leukemia (AMoL). In one embodiment, the lymphoma is follicular lymphoma, Hodgkin's lymphoma, or Non-Hodgkin's lymphoma, optionally wherein the Hodgkin's lymphoma is Nodular sclerosing subtype, mixed-cellularity subtype, lymphocyte-rich subtype, or lymphocyte depleted subtype. In one embodiment, the cancer is a solid cancer comprising melanoma, unresectable melanoma, metastatic melanoma, renal cancer, renal cell carcinoma, prostate cancer, metastatic castration resistant prostate cancer, ovarian cancer, epithelial ovarian cancer, metastatic epithelial ovarian cancer, breast cancer, triple negative breast cancer, lung cancer, and/or non-small cell lung cancer. In one embodiment, the cancer is melanoma. In one embodiment, the cancer is ovarian cancer.

In another aspect the present invention provides a method of treating an inflammatory disease or disorder in a subject, the method comprising administering to the subject an adoptive cell therapeutic composition; and an antagonist of collagen or a functional fragment thereof, thereby treating the autoimmune disease or disorder in the subject.

In one embodiment, the adoptive cell therapeutic composition comprises a cell type selected from a group consisting of a tumor infiltrating lymphocytes (TIL), T-cell receptor modified lymphocytes, and chimeric antigen receptor modified lymphocytes. In one embodiment, the adoptive cell therapeutic composition comprises tumor infiltrating lymphocytes (TIL). In one embodiment, the adoptive cell therapeutic composition comprises a cell type selected from a group consisting of T-cells, CD8+ cells, CD4+ cells, NK-cells, delta-gamma T-cells, regulatory T-cells, and peripheral blood mononuclear cells. In one embodiment, the adoptive cell therapeutic composition comprises T-cells.

In one embodiment, the collagen comprises collagen type-I, collagen type II, collagen type III, or collagen type-IV. In one embodiment, the antagonist of collagen and functional fragment thereof comprises an antagonist of collagen type-I or a functional fragment thereof, or an antagonist of collagen type-IV or a functional fragment thereof. In one embodiment, the antagonist of collagen type-I or the functional fragment thereof comprises an antagonist of the XL313 cryptic collagen epitope, or an antagonist of the HU177 cryptic collagen epitope. In one embodiment, the antagonist of collagen type-I or the functional fragment thereof comprises an antibody or an antigen-binding fragment thereof that binds a cryptic RGDKGE (SEQ ID NO: 1) containing collagen epitope. In one embodiment, the antagonist of collagen type-I or the functional fragment thereof comprises an antibody or an antigen-binding fragment thereof that binds a cryptic CPGFPGFC (SEQ ID NO: 16) containing collagen epitope. In one embodiment, the antibody or the antigen-binding fragment thereof comprises a monoclonal antibody or an antigen-binding fragment thereof. In one embodiment, the monoclonal antibody or antigen-binding fragment thereof comprises an XL313 monoclonal antibody or an antigen-binding fragment thereof. In one embodiment, the monoclonal antibody or antigen-binding fragment thereof comprises an HU177 monoclonal antibody or an antigen-binding fragment thereof.

In one embodiment, the migration of T-cells to the inflammation in the subject is decreased. In one embodiment, the migration of CD8+ T-cells to inflammation in the subject is increased. In one embodiment, the migration of CD4+ Treg-cells and/or MDSCs to inflammation in the subject is decreased.

In one embodiment, the level of phosphorylation and/or activation of a P38 MAP Kinase in the subject is inhibited compared to a control.

In one embodiment, the methods provided herein further comprise administering an antagonist of an integrin to the subject. In one embodiment, the integrin comprises integrin αvβ3. In one embodiment, the antagonist of integrin αvβ3 comprises an antibody capable of binding an RGDKGE (SEQ ID NO: 1) containing collagen epitope, or a CPGFPGFC (SEQ ID NO: 16) containing collagen epitope.

In one embodiment, the adoptive cell therapeutic composition and the antagonist of collagen or the functional fragment thereof are administered simultaneously to the subject. In one embodiment, the adoptive cell therapeutic composition and the antagonist of collagen or the functional fragment thereof are administered consecutively, in any order, to the subject. In one embodiment, the adoptive cell therapeutic composition, the antagonist of collagen or the functional fragment thereof, and the antagonist of an integrin are administered simultaneously to the subject. In one embodiment, the adoptive cell therapeutic composition, the antagonist of collagen or the functional fragment thereof, and the antagonist of an integrin to the subject is conducted consecutively, in any order. In one embodiment, there is a time period of one minute to four weeks between the consecutive administration of the adoptive cell therapeutic composition and the antagonist of collagen or the functional fragment thereof and/or there are several administrations of the adoptive cell therapeutic composition and the antagonist of collagen or the functional fragment thereof. In one embodiment, the administration of the adoptive cell therapeutic composition and/or the antagonist of collagen or the functional fragment thereof is conducted through an intra-tumoral, intra-arterial, intravenous, intrapleural, intravesicular, intracavitary or peritoneal injection, or an oral administration.

In one embodiment, the inflammatory disease or disorder is selected from the group consisting of an allergy, ankylosing spondylitis, asthma, atopic dermatitis, an autoimmune disease or disorder, a cancer, celiac disease, chronic obstructive pulmonary disease (COPD), chronic peptic ulcer, cystic fibrosis, diabetes, glomerulonephritis, gout, hepatitis, an immune-mediated disease or disorder, inflammatory bowel disease (IBD), myositis, osteoarthritis, pelvic inflammatory disease (PID), multiple sclerosis, neurodegenerative diseases of aging, a periodontal disease, reperfusion injury transplant rejection, psoriasis, pulmonary fibrosis, rheumatic disease, scleroderma, sinusitis, dermatitis, pneumonitis, colitis and tuberculosis. In one embodiment, the inflammatory disease or disorder is an autoimmune disease or disorder. In one embodiment, the autoimmune disease or disorder is selected from the group consisting of Psoriasis, Graft-vs-Host Disease, Amyotrophic Lateral Sclerosis, Pemphigus Vulgaris, Systemic Lupus Erythematosus, Scleroderma, Ulcerative Colitis, Crohn's Disease, Type 1 Diabetes, Multiple Sclerosis, Alopecia Areata, Uveitis, Neuromyelitis Optica, Graves' disease, Hashimoto's thyroiditis, rheumatoid arthritis and Duchenne Muscular Dystrophy.

In another aspect, the present invention provides a pharmaceutical kit comprising an adoptive cell therapeutic composition and an antagonist of collagen or a functional fragment thereof, wherein the adoptive cell therapeutic composition is formulated in a first formulation and the antagonist of collagen or the functional fragment thereof are formulated in a second formulation. In one embodiment, the pharmaceutical kit as provided herein, further comprises an antagonist of an integrin, wherein the antagonist of an integrin is formulated in a third formulation. In one embodiment, the first formulation and the second formulation and/or third formulation are for simultaneous or sequential, in any order, administration to a subject.

### BRIEF DESCRIPTION OF THE DRAWINGS

FIG. 1. Selective expression of proteolyzed/denatured forms of collagen associated with tumor vasculature. Frozen sections of malignant melanomas were blocked with BSA to limit non-specific binding. Next, tumor tissues were co-stained with anti-CD31 antibody to mark blood vessels (red) and anti-HU177 antibody directed to denatured forms of collagen (green). Examples of colocalization of denatured collagen (green) with tumor blood vessels (red) in B16F10 melanoma tumors. Photos were taken at a magnification of 400×.

FIGS. 2A-2F. Detection of RGDKGE (SEQ ID NO: 1)-containing collagen peptide in malignant tumor cells and expression of key signaling molecules in T-cells. Briefly, whole cell lysates and serum free concentrated conditioned medium was prepared from tumor cells lines. FIG. 2A. Western blot analysis of whole cell lysates from human C8161 and murine B16F10 melanoma cells for the expression of the low molecular weight 16 kDa RGDKGE (SEQ ID NO: 1)-containing collagen peptide (XL313) or actin as a loading control. FIG. 2B. Western blot analysis of concentrated serum free conditioned medium from B16F10 melanoma cells for the expression of the low molecular weight 16 kDa RGDKGE (SEQ ID NO: 1)-containing collagen fragment (XL313). FIG. 2C. Western blot analysis of whole cell lysates from human Jurkat T-cells for the expression of the CD8, β3 integrin and control protein actin. FIG. 2D. Western blot analysis of whole cell lysates from murine primary CD8+ T-cells for the expression of the CD8, β3 integrin and control protein actin. FIG. 2E. Western blot analysis of whole cell lysates from human Jurkat T-cells following stimulation with non-specific control peptide CP or the RGDKGE (SEQ ID NO: 1)-containing collagen peptide P2 for the expression of the phosphorylated (P-β3 Integrin) and total β3 integrin (T-β3 Integrin) and control protein actin. FIG. 2F. Western blot analysis of whole cell lysates from murine primary CD8+ T-cells following stimulation with non-specific control peptide CP or the RGDKGE (SEQ ID NO: 1)-containing collagen peptide P2 for the expression of the phosphorylated and total β3 integrin and control protein actin.

FIGS. 3A-3C. The RGDKGE (SEQ ID NO: 1)-containing collagen peptide P2 inhibits CD8+ T-cell polarization on denatured collagen type-IV. Briefly, microscopy slides were coated with denatured collagen type-IV to mimic the altered subendothelial basement membranes of tumor associated blood vessels. Murine primary CD8+ T-cells were isolated from mice bearing B16F10 melanomas and stimulated with either non-specific control peptide CP or the RGDKGE (SEQ ID NO: 1)-containing collagen peptide P2 and seeded on the coated slides. T-cells were allowed to incubate for 1 hour. Next slides were washed, fixed and stained with Rhodamine-labeled Phalloidin to mark F-actin. Cells were analyzed for morphological evidence of cell shape changes and polarized F-actin. FIG. 3A. Example of an enlarged image of CD8+ T-cell morphology following stimulation with either non-specific control peptide CP or the RGDKGE (SEQ ID NO: 1)-containing collagen peptide P2. FIG. 3B. Example of CD8+ T-cell morphology following stimulation with either non-specific control peptide CP or the RGDKGE (SEQ ID NO: 1)-containing collagen peptide P2. White arrows indicate examples of cells exhibiting polarization. FIG. 3C. Quantification of the mean percentage of polarized CD8+ T-cells per 400× microscopic field. Data bars indicate mean percentage of polarized cells per 400× microscopic field from two independent experiments with 10 to 11 fields per treatment. *P<0.05.

FIGS. 4A-4H. The RGDKGE (SEQ ID NO: 1)-containing collagen peptide P2 selectively inhibits T-cell migration through denatured collagen type-IV. Briefly, membranes from transwell migration chambers were coated with either native collagen type-IV or denatured collagen type-IV. T-cell variants were incubated with non-specific control peptide CP, or the RGDKGE (SEQ ID NO: 1)-containing collagen peptide (P2) and seed on the coated transwell membranes. T-cells were allowed to migrate in the presence or absence of SDF-1 added to the lower chamber for approximately 1.5 hours. Cell migration was quantified by counting the number of crystal violet stained cells that had migrated through to the underside of the coated membrane. FIG. 4A. Jurkat T-cell migration through denatured collagen type-IV in the presence of non-specific control peptide CP or RGDKGE (SEQ ID NO: 1)-containing collagen peptide P2 with SDF-1. FIG. 4B. Jurkat T-cell migration through native collagen type-IV in the presence of non-specific control peptide CP or RGDKGE (SEQ ID NO: 1)-containing collagen peptide P2 with SDF-1. FIG. 4C. Cem/C1 T-cell migration through denatured collagen type-IV in the presence of non-specific control peptide CP or RGDKGE (SEQ ID NO: 1)-containing collagen peptide P2 with SDF-1. FIG. 4D. Cem/C1 T-cell migration through native collagen type-IV in the presence of non-specific control peptide CP or RGDKGE (SEQ ID NO: 1)-containing collagen peptide P2. FIG. 4E. Hut78 T-cell migration through denatured collagen type-IV in the presence of non-specific control peptide CP or RGDKGE (SEQ ID NO: 1)-containing collagen peptide P2. FIG. 4F. Hut78 T-cell migration through native collagen type-IV in the presence of non-specific control peptide CP or RGDKGE (SEQ ID NO: 1)-containing collagen peptide P2. FIG. 4G Primary CD8+ T-cell migration through denatured collagen type-IV in the presence of non-specific control peptide CP or RGDKGE (SEQ ID NO: 1)-containing collagen peptide P2 with SDF-1. FIG. 4H. CD8+ T-cell migration through native collagen type-IV in the presence of non-specific control peptide CP or RGDKGE (SEQ ID NO: 1)-containing collagen peptide P2 with SDF-1. Data bars indicate the mean cell migration±S.E. *P<0.05.

FIGS. 5A-5F. Mab XL313 selectively enhances T-cell migration through denatured collagen type-IV. Briefly, membranes from transwell migration chambers were coated with denatured collagen type-IV. T-cell variants were incubated with the RGDKGE (SEQ ID NO: 1)-containing collagen peptide (P2) or B16F10 melanoma cell conditioned medium as a source of the RGDKGE (SEQ ID NO: 1)-containing collagen peptide and seeded on the transwell membranes in the presence of non-specific control antibody or Mab XL313 specifically directed to the RGDKGE (SEQ ID NO: 1) collagen peptide. T-cells were allowed to migrate in the presence of SDF-1 for approximately 1.5 hours. Cell migration was quantified by counting the number of crystal violet stained cells that had migrated through to the underside of the coated membrane. FIG. 5A. Jurkat T-cell migration through denatured collagen type-IV in the presence of non-specific control antibody (Ab Cont) or Mab XL313. FIG. 5B. Jurkat T-cell migration through native collagen type-IV in the presence of non-specific control antibody (Ab Cont) or Mab XL313. FIG. 5C. Jurkat T-cell migration in presence of B16F10 condition medium through denatured collagen type-IV in the presence of non-specific control antibody (Ab Cont) or Mab XL313. FIG. 5D. Jurkat T-cell migration through native collagen type-IV in presence of B16F10 conditioned medium in the presence of non-specific control antibody (Ab Cont) or Mab XL313. FIG. 5E. CD8+ T-cell migration through denatured collagen type-IV in the presence of non-specific control antibody (Ab Cont) or Mab XL313. FIG. 5F. CD8+ T-cell migration through denatured collagen type-IV in the presence of non-specific control antibody (Ab Cont) or Mab XL313 using B16F10 conditioned medium as the source of the collagen peptide. Data bars indicate the mean cell migration±S.E. *P<0.05.

FIGS. 6A-6E. Function blocking anti-CD8a antibody depletes CD8+ T-cells in C57BL/6 mice leading to enhanced B16F10 tumor growth. Briefly, C57BL/6 mice were treated 2× per week with anti-mouse CD8 antibody or non-specific control antibody. At the end of the treatment protocol, mice were sacrificed and blood and organs were collected for analysis. FIG. 6A. Example of flow cytometry for the presence of CD8+ T-cells in the blood of mice from each condition. FIG. 6B. Example of quantification of flow cytometry analysis for the presence of CD8+ T-cells in blood from mice from each condition. FIG. 6C. Example of quantification of flow cytometry analysis for the presence of CD4+ T-cells and F/480 macrophages in blood from mice from each condition. To examine the role of CD8+ T-cells to regulate B16F10 tumor growth, mice were first depleted of CD8+ T-cells as previously described. Next, control depleted or CD8+ T-cell depleted mice were injected with B16F10 melanoma cells and allowed to grow for 14 days. FIG. 6D. Example of B16F10 melanoma tumors dissected from mice from each condition. FIG. 6E. Quantification of mean B16F10 tumor growth from each condition. Data bars represent mean tumor volume±S.E. from each condition. *P<0.05.

FIGS. 7A-7C. Mab XL313 fails to inhibit B16F10 tumor growth in mice depleted of CD8+ T-cells. To determine the functional role of CD8+ T-cells in the ability of Mab XL313 to control tumor growth, we depleted CD8+ T-cells from C57BL/6 mice as we described above. Briefly control depleted mice and CD8+ T-cell depleted mice were injected with B16F10 melanoma cells and treated with Mab XL313 or non-specific control antibody 3× per week. At the end of the treatment period, mice were sacrificed and tumor growth quantified. FIG. 7A. Example of treatment protocol. FIG. 7B. Examples of the size of B16F10 tumors from mice from each experimental condition on day 14. FIG. 7C. Quantification of B16F10 tumor growth from each condition on day 14. Data points indicate tumor volume from each tumor from mice from each condition on day 14.

FIGS. 8A-8C. Mab XL313 enhances CD8+ T-cell of infiltration of tumors growing in vivo. Given our surprising findings that the RGDKGE (SEQ ID NO: 1) collagen peptide can selectively inhibit T-cell migration though denatured collagen type-IV, but not native collagen type-IV, and that Mab XL313 directed to the RGDKGE (SEQ ID NO: 1) collagen peptide can selectively enhance T-cell migration through denatured collagen type-IV, but not native collagen-IV in vitro, we sought to test the effects of Mab XL313 on CD8+ T-cell infiltration of tumors in vivo. Briefly, B16F10 melanoma cell were injected in mice and allowed to growth. Mice with B16F10 tumors were treated 3× per week with Mab XL313 or non-specific control antibody. At the end of the treatment period (14 days), mice were sacrificed, tumors dissected, measured, and single cell suspensions were prepared for each tumor and analyzed by flow cytometry for the levels of infiltrating CD8+ T-cells. FIG. 8A. Quantification of mean B16F10 tumor size from each condition. FIG. 8B. Quantification of mean CD8+ T-cell infiltration of B16F10 tumors from each condition. FIG. 8C. SKOV-3 tumor cells were injected in humanized mice and then treated 3× per week with Mab XL313 or non-specific control antibody. At the end of the treatment period (21 days), mice were sacrificed, tumors dissected, measured, and single cell suspensions were prepared for each tumor and analyzed by flow cytometry for the levels of infiltrating CD8+ T-cells. Data bars represent mean±S.E. CD8+ T-cell infiltration as a percentage of control with 5-6 mice per condition.

FIGS. 9A-9C. T-cell lines were allowed to migrate in the presence of the chemoattractant SDF-1 in the lower chamber. As shown in FIG. 9A and FIG. 9B, migration of human T-cell lines Jurkat and Hut78 were significantly reduced on denatured collagen type-IV as compared to native collagen type-IV. FIG. 9C depicts isolated primary T-cells from B16F10 tumor bearing mice also showed a similar reduction in migratory ability on denatured collagen type-IV.

FIG. 10 depicts Mab HU177 significantly enhanced Hut78 T-cell migration on denatured collagen type-IV.

FIG. 11 shows that anti-HU177 antibody significantly increased the levels of CD8+ T-cells in ascites fluid from mice with ID8-VEGF ovarian tumors. Data bars represent the mean percentage of CD8+ T-cells (+SE from 4 mice per condition) in the ascites fluid from each experimental group.

FIG. 12A shows that CD4+ Treg cells isolated from B16F10 tumor bearing mice exhibit enhanced migration on denatured collagen-IV as compared to native collagen-IV. Briefly, mice (C57BL/6) were injected subcutaneously with B16F10 cells. B16F10 tumors were allowed to grow for 14-days to allow formation of solid malignant melanomas. Spleens from the tumor bearing mice were dissected and primary murine CD4+ Treg cells were isolated and analyzed for migration on either native collagen type-IV or denatured collagen type-IV. Data bars represent the mean CD4+ Treg cell migration+SE from 3 wells per condition. FIG. 12B shows that CD4+ Treg cells isolated from ID8-VEGF tumor bearing mice exhibit enhanced migration on denatured collagen-IV as compared to native collagen-IV. Briefly, mice (C57BL/6) were injected intraperitoneally with ID8-VEGF cells. ID8-VEGF tumors were allowed to grow for 14-days. Spleens from the tumor bearing mice were dissected and primary murine CD4+ Treg cells were isolated and analyzed for migration on either native collagen type-IV or denatured collagen type-IV. Data bars represent the mean CD4+ Treg cell migration+SE from 3 wells per condition.

FIG. 13 shows that stimulation of CD4+ Treg cells with the soluble RGDKGE (SEQ ID NO: 1) collagen peptide (P2) enhances migration on denatured collagen-IV as compared to stimulation with control peptide (CP). Briefly, mice (C57BL/6) were injected subcutaneously with B16F10 cells. B16F10 tumors were allowed to grow for 14-days to allow formation of solid malignant melanomas. Spleens from tumor bearing mice were dissected and primary murine CD4+ Treg cells were isolated and analyzed for the effects of the soluble collagen peptide (P2) or control peptide (CP) on cell migration on denatured collagen type-IV. Data bars represent mean CD4+ Treg cell migration+SE from 3 wells per condition.

FIG. 14 shows that the anti-HU177 antibody reduces the levels of CD4+ Treg cells in ascites fluid from mice with ID8-VEGF ovarian tumors. Mice (C57BL/6) were injected intraperitoneally with ID8-VEGF ovarian tumor cells. ID8-VEGF tumor cells were allowed to grow within the peritoneal cavity of mice for 4-weeks to allow formation of malignant ascites fluid and solid tumors. Beginning at 4 weeks, mice were injected subcutaneously with 100 μg per mouse 2× per week with non-specific control antibody (Ab Cont) or anti-HU177 antibody (Anti-HU177). At 12 weeks, mice were sacrificed and malignant ascites fluid was collected. The relative levels of CD4+ Treg-cells within the malignant ascites fluid was quantified by flow cytometry. Data bars represent the mean percentage of CD4+ Treg cells+SE from 4 mice per condition in the ascites fluid from each experimental group.

FIG. 15A shows that reduced levels of Myeloid-Derived Suppressor Cells (MDSC) were detected in B16F10 melanoma tumors growing in α10 integrin knockdown mice. Briefly, wildtype α10 integrin expressing mice (WT) and α10 integrin knockdown mice (α10-K/D) were injected with equal numbers of B16F10 melanoma cells and allowed to form tumor for 14 days. Tumors were then dissected and single cell suspensions were prepared and analyzed for the relative levels of MDSC from each condition. Data bars represent the mean relative percentage of MDSC within tumor tissues+SE from 4 mice per condition. FIG. 15B shows that enhanced levels of CD8+ T-cells detected in B16F10 melanoma tumors growing in α10 integrin knockdown mice. Briefly, wildtype α10 integrin expressing mice (WT) and α10 integrin knockdown mice (α10-K/D) were injected with equal numbers of B16F10 melanoma cells and allowed to form tumor for 14 days. Tumors were then dissected and single cell suspensions were prepared and analyzed for the relative levels of CD8+ T-cells from each condition. Data bars represent the mean relative percentage of CD8+ T-cells within tumor tissues+SE from 3 to 4 mice per condition.

FIG. 16 shows that selective targeting of the RGDKGE (SEQ ID NO: 1)-containing collagen peptide P2 inhibits the levels of activated/phosphorylated P38MAPK. The RGDKGE (SEQ ID NO: 1) collagen peptide P2 was pre-incubated with non-specific control antibody (Ab Control) or anti-RGDKGE (SEQ ID NO: 1) (SEQ ID NO: 1) Mab XL313 to inhibit P2 for 30 minutes. Jurkat T-cells were stimulated under each of these conditions for 15 minutes, and whole cell lysates were prepared and analyzed for levels of phosphorylated P38MAPK by Western blotting. Tubulin was used as a loading control. P-p38 and T-p38 refer to phosphorylated (activated) and total P38MAPK, respectively.

### DETAILED DESCRIPTION

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein “adoptive cell therapeutic composition” refers to any composition comprising cells suitable for adoptive cell transfer. In one embodiment of the invention the adoptive cell therapeutic composition comprises a cell type selected from a group consisting of a tumor infiltrating lymphocyte (TIL), TCR (i.e. heterologous T-cell receptor) modified lymphocytes and CAR (i.e. chimeric antigen receptor) modified lymphocytes. In another embodiment of the invention, the adoptive cell therapeutic composition comprises a cell type selected from a group consisting of T-cells, CD8+ cells, CD4+ cells, NK-cells, delta-gamma T-cells, regulatory T-cells and peripheral blood mononuclear cells. In another embodiment, TILs, T-cells, CD8+ cells, CD4+ cells, NK-cells, delta-gamma T-cells, regulatory T-cells or peripheral blood mononuclear cells form the adoptive cell therapeutic composition. In one specific embodiment of the invention the adoptive cell therapeutic composition comprises T cells. As used herein “tumor-infiltrating lymphocytes” or TILs refer to white blood cells that have left the bloodstream and migrated into a tumor. Lymphocytes can be divided into three groups including B cells, T cells and natural killer cells. In another specific embodiment of the invention the adoptive cell therapeutic composition comprises T-cells which have been modified with target-specific chimeric antigen receptors or specifically selected T-cell receptors. As used herein “T-cells” refers to CD3+ cells, including CD4+ helper cells, CD8+ cytotoxic T-cells and γδ T cells.

By “agent” is meant any small molecule chemical compound, antibody, nucleic acid molecule, or polypeptide, or fragments thereof.

The term “antagonist” refers to a moiety that prevents a biomolecule, e.g., a protein, nucleic acid, or ribozyme, from completing a reaction, renders the active agent unavailable to produce a pharmacological effect, inhibits the function of an agonist, or produces an adverse pharmacological effect. Antagonists can compete with an agonist for a specific binding site (competitive antagonists) and/or can bind to a different binding site from the agonist, hindering the effect of the agonist via the other binding site (non-competitive antagonists). Non-limiting examples of antagonists include nucleic acid, protein, or small molecule inhibitors. In some embodiments, an antagonist of a biomolecule is an antibody that recognizes and binds to the biomolecule. In some embodiments, the antagonist binds a biomolecule and block binding of a binding partner (e.g., a protein, nucleic acid, or ribozyme).

By “antibody” is meant any immunoglobulin polypeptide, or fragment thereof, having immunogen binding ability. As used herein, the term “antibodies” includes polyclonal antibodies, affinity-purified polyclonal antibodies, monoclonal antibodies, and antigen-binding fragments, such as F(ab′)2 and Fab proteolytic fragments. Genetically engineered intact antibodies or fragments, such as chimeric antibodies, Fv fragments, single chain antibodies, and the like, as well as synthetic antigen-binding peptides and polypeptides, are also included. Non-human antibodies may be humanized by grafting non-human CDRs onto human framework and constant regions, or by incorporating the entire non-human variable domains. In certain preferred embodiments, humanized antibodies may retain non-human residues within the human variable region framework domains to enhance proper binding characteristics.

“Antigenic fragment” and the like are understood as at least that portion of a peptide capable of inducing an immune response in a subject, or being able to be specifically bound by an antibody raised against the antigenic fragment. Typically, antigenic fragments are at least 7 amino acids in length. Antigenic fragments can include deletions of the amino acid sequence from the N-terminus or the C-terminus, or both. For example, an antigenic fragment can have an N- and/or a C-terminal deletion of about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, or more amino acids. Antigenic fragments can also include one or more internal deletions of the same exemplary lengths. Antigenic fragments can also include one or more point mutations, particularly conservative point mutations. At least an antigenic fragment of protein can include the full length, wild-type sequence of the antigen. An antigenic fragment can include more than one potential antibody binding site. An antigenic fragment can be used to make antibodies for use in any of the methods provided herein.

By “autoimmune disease” is meant a disease characterized by a dysfunction in the immune system. The disease is characterized by the components of the immune system affected, whether the immune system is overactive or underactive, or whether the condition is congenital or acquired. In most cases, the disorder causes abnormally low activity or over activity of the immune system. In cases of immune system over activity, the body attacks and damages its own tissues (autoimmune). Immune deficiency diseases decrease the body's ability to fight invaders, causing vulnerability to infections. In response to an unknown trigger, the immune system may begin producing antibodies that instead of fighting infections, attack the body's own tissues. Treatment for autoimmune diseases generally focuses on reducing immune system activity.

By “blood vessel formation” is meant the dynamic process that includes one or more steps of blood vessel development and/or maturation, such as angiogenesis, arteriogenesis, vasculogenesis, formation of an immature blood vessel network, blood vessel remodeling, blood vessel stabilization, blood vessel maturation, blood vessel differentiation, or establishment of a functional blood vessel network.

By “blood vessel remodeling” or “vascular remodeling” is meant the dynamic process of blood vessel enlargement in shape and size to maintain the luminal orifice and blood flow. For example, vascular remodeling includes change in arterial size to adapt to plaque accumulation, effectively maintaining the lumen and blood flow to the myocardium.

As used herein, “binding” or “specific binding” is understood as having at least a 10³ or more, preferably 10⁴ or more, preferably 10⁵ or more, preferably 10⁶ or more preference for binding to a specific binding partner as compared to a non-specific binding partner (e.g., binding an antigen to a sample known to contain the cognate antibody).

By “cancer” is meant, comprising of but not limited to melanoma, central nervous system (CNS) cancer, CNS germ cell tumor, lung cancer, leukemia, multiple myeloma, renal cancer, malignant glioma, medulloblatoma, breast cancer, ovarian cancer, prostate cancer, bladder cancer, fibrosarcoma, pancreatic cancer, gastric cancer, head and neck cancer, colorectal cancer. For example, a cancer cell is derived from a solid cancer or hematological cancer. The hematological cancer is, e.g., a leukemia or a lymphoma. A leukemia is acute lymphoblastic leukemia (ALL), acute myelogenous leukemia (AML), chronic lymphocytic leukemia (CLL), small lymphocytic lymphoma (SLL), chronic myelogenous leukemia (CML), or acute monocytic leukemia (AMoL). A lymphoma is follicular lymphoma, Hodgkin's lymphoma (e.g., Nodular sclerosing subtype, mixed-cellularity subtype, lymphocyte-rich subtype, or lymphocyte depleted subtype), or Non-Hodgkin's lymphoma. Exemplary solid cancers include but are not limited to melanoma (e.g., unresectable, metastatic melanoma), renal cancer (e.g., renal cell carcinoma), prostate cancer (e.g., metastatic castration resistant prostate cancer), ovarian cancer (e.g., epithelial ovarian cancer, such as metastatic epithelial ovarian cancer), breast cancer (e.g., triple negative breast cancer), and lung cancer (e.g., non-small cell lung cancer).

By “control” or “reference” is meant a standard of comparison. As used herein, “changed as compared to a control” sample or subject is understood as having a level of the analyte or diagnostic or therapeutic indicator to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection the amount and measurement of the change can vary. Determination of statistical significance is within the ability of those skilled in the art, e.g., the number of standard deviations from the mean that constitute a positive result. In some embodiments, the control or reference is from the subject prior to administration of the therapeutic as presently described herein. In some embodiments, the control of reference comprises a standard derived from efficacy measurements of adoptive cell therapeutic composition administered to subjects without an antagonist of collagen or a functional fragment thereof. In some embodiments, the control or reference is a measurement of T-cell infiltration into a tumor in the subject prior to the administration of the combination of an antagonist of collagen or a functional fragment thereof and an adoptive cell therapeutic composition. In some embodiments, the control or reference is a measurement of Treg and/or myeloid-derived suppressor cell (MDSC) levels in a tumor in the subject prior to the administration of the combination of an antagonist of collagen or a functional fragment thereof and an adoptive cell therapeutic composition.

By “cryptic” is meant that a motif may be inaccessible to cell surface receptors, and once the target protein is proteolyzed or denatured, a sequence becomes exposed or generates a fragment that is then recognized by the antibody. For example, the XL313 epitope, i.e., RGDKGE (SEQ ID NO: 1) core sequence within collagen type-I is cryptic in that the antibody does not react with normal collagen in its triple helical state, but once it is proteolyzed or denatured, the sequence becomes exposed or generates a fragment of collagen that is recognized by Mab XL313. Similarly, the HU177 epitope, i.e., CPGFPGFC (SEQ ID NO: 16) core sequence within collagen type-I is cryptic in that the antibody does not react with normal collagen in its triple helical state, but once it is proteolyzed or denatured, the sequence becomes exposed or generates a fragment of collagen that is recognized by Mab HU177.

As used herein, “detecting”, “detection” and the like are understood that an assay performed for identification of a specific analyte in a sample, e.g., an antigen in a sample or the level of an antigen in a sample. The amount of analyte or activity detected in the sample can be none or below the level of detection of the assay or method.

By “diagnosing” and the like as used herein refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. Diagnostic methods provide an indicator that a disease is or is not present. A single diagnostic test typically does not provide a definitive conclusion regarding the disease state of the subject being tested.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “functional fragment” or “fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full length or wild-type protein. In some embodiments, the functional fragment may have a reduced biological activity, about equivalent biological activity, or an enhanced biological activity as compared to the wild-type or full-length polypeptide sequence upon which the fragment is based.

In some embodiments, the functional fragment may retain 99%, 98%, 97%, 96%, 95%, 94%, 93%, 92%, 91%, or 90% sequence identity to the wild-type sequence upon which the sequence is derived. In some embodiments, the functional fragment may retain 85%, 80%, 75%, 70%, 65%, or 60% sequence homology to the wild-type sequence upon which the sequence is derived.

The term “polynucleotide” or “nucleic acid” as used herein designates mRNA, RNA, cRNA, cDNA or DNA. As used herein, a “nucleic acid encoding a polypeptide” is understood as any possible nucleic acid that upon (transcription and) translation would result in a polypeptide of the desired sequence. The degeneracy of the nucleic acid code is well understood. Further, it is well known that various organisms have preferred codon usage, etc. Determination of a nucleic acid sequence to encode any polypeptide is well within the ability of those of skill in the art.

As used herein, “immunoassay” is understood as any antibody base detection method including, but not limited to enzyme linked immunosorbent assay (ELISA), radioimmune assay (RIA), Western blot, immunohistochemistry, immunoprecipitation assay such as Luciferase Immunoprecipitation System (LIPS see, e.g., US Patent Publication 2007/0259336 which is incorporated herein by reference). In a preferred embodiment, the immunoassay is a quantitative. Antibodies for use in immunoassays include any monoclonal or polyclonal antibody appropriate for use in the specific immunoassay.

By “inhibitory nucleic acid molecule” is meant a polynucleotide that disrupts the expression of a target nucleic acid molecule or an encoded polypeptide. Exemplary inhibitory nucleic acid molecules include, but are not limited to, shRNAs, siRNAs, antisense nucleic acid molecules, and analogs thereof.

As used herein, “isolated” or “purified” when used in reference to a polypeptide means that a naturally polypeptide or protein has been removed from its normal physiological environment (e.g., protein isolated from plasma or tissue, optionally bound to another protein) or is synthesized in a non-natural environment (e.g., artificially synthesized in an in vitro translation system or using chemical synthesis). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

As used herein, “kits” are understood to contain at least one non-standard laboratory reagent for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

As used herein, “obtaining” is understood herein as manufacturing, purchasing, or otherwise coming into possession of.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Formulations of the present invention include those suitable for oral, nasal, topical, transdermal, buccal, sublingual, intramuscular, intracardiac, intraperotineal, intrathecal, intracranial, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments). Optionally the peptide further includes one or more modifications such as modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins, Structure and Molecular Properties, 2nd ed., T. E. Creighton, W.H. Freeman and Company, New York (1993); Posttranslational Covalent Modification of Proteins, B. C. Johnson, ed., Academic Press, New York, pgs. 1-12 (1983); Seifter et al., Meth. Enzymol 182:626-646 (1990); Rattan et al., Ann. N.Y. Acad. Sci. 663:48-62 (1992)).

The terms “reduce” or “inhibit” refer to altering negatively by at least 5%. The terms “increase” or “enhance” refer to altering positively by at least 5%. The term “modulate” refers to altering negatively or positively by at least 5%. An alteration may be by 5%, 10%, 25%, 30%, 50%, 75%, or even by 100%.

As used herein, a “reporter protein” or a “reporter polypeptide” is understood as a polypeptide that can be readily detected, preferably quantitatively detected, either directly or indirectly. A reporter polypeptide typically has an enzymatic activity, luciferase activity, alkaline phosphatase activity, beta-galactosidase activity, acetyl transferase activity, etc. wherein catalysis of a reaction with the substrate by the enzyme results in the production of a product, e.g., light, a product that can be detected at a specific wavelength of light, radioactivity, such that the amount of the reporter peptide can be determined in the sample, either as a relative amount, or as an absolute amount by comparison to control samples.

A “sample” as used herein refers to a biological material that is isolated from its environment (e.g., blood or tissue from an animal, cells, or conditioned media from tissue culture) and is suspected of containing, or known to contain an analyte, such as a protein. A sample can also be a partially purified fraction of a tissue or bodily fluid. A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

“Sensitivity and specificity” are statistical measures of the performance of a binary classification test. The sensitivity (also called recall rate in some fields) measures the proportion of actual positives which are correctly identified as such (e.g. the percentage of sick people who are identified as having the condition); and the specificity measures the proportion of negatives which are correctly identified (e.g. the percentage of well people who are identified as not having the condition). They are closely related to the concepts of type I and type II errors. A theoretical, optimal prediction can achieve 100% sensitivity (i.e. predict all people from the sick group as sick) and 100% specificity (i.e. not predict anyone from the healthy group as sick).

The concepts are expressed mathematically as follows:
sensitivity=#true positives/#true positives+#false negatives
specificity=#true negatives/#true negatives+#false positives.

By “selectively” is meant the ability to affect the activity or expression of a target molecule without affecting the activity or expression of a non-target molecule.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST®, BESTFIT™ GAP™, or PILEUP/PRETTYBOX™ programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST® program may be used, with a probability score between e-3 and e-100 indicating a closely related sequence.

A “subject” as used herein refers to an organism. In certain embodiments, the organism is an animal. In certain embodiments, the subject is a living organism. In certain embodiments, the subject is a cadaver organism. In certain preferred embodiments, the subject is a mammal, including, but not limited to, a human or non-human mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A “subject sample” can be a sample obtained from any subject, typically a blood or serum sample, however the method contemplates the use of anybody fluid or tissue from a subject. The sample may be obtained, for example, for diagnosis of a specific individual for the presence or absence of a particular disease or condition.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with diminished cardiac function is within the ability of those in the art. Subjects suffering from, and suspected of suffering from, a specific disease, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. Therapeutic effect may be assessed by monitoring the symptoms of a patient, tumor markers in blood or for example a size of a tumor or the length of survival of the patient

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Suitable types of collagen include collagen type-I, collagen type-II, collagen type-III and collagen type-IV (e.g., the alpha 6 chain of collagen type-IV). In some cases, the antagonist of collagen or a fragment thereof comprises an antagonist of the XL313 cryptic collagen epitope or an antagonist of the HU177 cryptic collagen epitope. For example, the antagonist of the XL313 cryptic collagen epitope comprises an antibody that binds a cryptic RGDKGE (SEQ ID NO: 1) containing collagen epitope or wherein the antagonist of the HU177 cryptic collagen epitope comprises an antibody that binds a cryptic CPGFPGFC (SEQ ID NO: 16)-containing collagen epitope. Preferably, the antibody comprises a monoclonal antibody, e.g., an XL313 monoclonal antibody or an HU177 monoclonal antibody.

Preferably, the antagonist of collagen or a fragment thereof enhances anti-tumor activity of the immune checkpoint inhibitor and inhibits an inflammatory disease or disorder. Exemplary inflammatory diseases or disorders include, but are not limited to, an allergy, ankylosing spondylitis, asthma, atopic dermatitis, an autoimmune disease or disorder, a cancer, celiac disease, chronic obstructive pulmonary disease (COPD), chronic peptic ulcer, cystic fibrosis, diabetes, glomerulonephritis, gout, hepatitis, an immune-mediated disease or disorder, inflammatory bowel disease (IBD), myositis, osteoarthritis, pelvic inflammatory disease (PID), multiple sclerosis, neurodegenerative diseases of aging, a periodontal disease, reperfusion injury transplant rejection, psoriasis, pulmonary fibrosis, rheumatic disease, scleroderma, sinusitis, dermatitis, pneumonitis, colitis and tuberculosis. In some embodiments, the inflammatory disease or disorder is an autoimmune disease or disorder. Exemplary autoimmune diseases or disorders include, but are not limited to, Psoriasis, Graft-vs-Host Disease, Amyotrophic Lateral Sclerosis, Pemphigus Vulgaris, Systemic Lupus Erythematosus, Scleroderma, Ulcerative Colitis, Crohn's Disease, Type 1 Diabetes, Multiple Sclerosis, Alopecia Areata, Uveitis, Neuromyelitis Optica, Graves' disease, Hashimoto's thyroiditis, rheumatoid arthritis and Duchenne Muscular Dystrophy.

The methods described herein can be used in conjunction with one or more chemotherapeutic or anti-neoplastic agents. In some cases, the additional chemotherapeutic agent is radiotherapy. In some cases, the chemotherapeutic agent is a cell death-inducing agent.

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

Exemplary cancers are selected from the group comprising of melanoma, central nervous system (CNS) cancer, CNS germ cell tumor, lung cancer, leukemia, multiple myeloma, renal cancer, malignant glioma, medulloblatoma, breast cancer, ovarian cancer, prostate cancer, bladder cancer, fibrosarcoma, pancreatic cancer, gastric cancer, head and neck cancer, colorectal cancer. For example, a cancer cell is derived from a solid cancer or hematological cancer. The hematological cancer is, e.g., a leukemia or a lymphoma. A leukemia is acute lymphoblastic leukemia (ALL), acute myelogenous leukemia (AML), chronic lymphocytic leukemia (CLL), small lymphocytic lymphoma (SLL), chronic myelogenous leukemia (CML), or acute monocytic leukemia (AMoL). A lymphoma is follicular lymphoma, Hodgkin's lymphoma (e.g., Nodular sclerosing subtype, mixed-cellularity subtype, lymphocyte-rich subtype, or lymphocyte depleted subtype), or Non-Hodgkin's lymphoma. Exemplary solid cancers include but are not limited to melanoma (e.g., unresectable, metastatic melanoma), renal cancer (e.g., renal cell carcinoma), prostate cancer (e.g., metastatic castration resistant prostate cancer), ovarian cancer (e.g., epithelial ovarian cancer, such as metastatic epithelial ovarian cancer), breast cancer (e.g., triple negative breast cancer), and lung cancer (e.g., non-small cell lung cancer).

Human dosage amounts can initially be determined by extrapolating from the amount of compound used in mice or nonhuman primates, as a skilled artisan recognizes it is routine in the art to modify the dosage for humans compared to animal models. In certain embodiments, it is envisioned that the dosage of the antagonist to collagen may vary from between about 0.1 μg compound/kg body weight to about 25000 μg compound/kg body weight; or from about 1 μg/kg body weight to about 4000 μg/kg body weight or from about g/kg body weight to about 3000 μg/kg body weight. In other embodiments this dose may be about 0.1, 0.3, 0.5, 1, 3, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000, 5500, 6000, 6500, 7000, 7500, 8000, 8500, 9000, 9500, 10000, 10500, 1100, 11500, 12000, 12500, 13000, 13500, 14000, 14500, 15000, 15500, 16000, 16500, 17000, 17500, 18000, 18500, 19000, 19500, 20000, 20500, 21000, 21500, 22000, 22500, 23000, 23500, 24000, 24500, or 25000 μg/kg body weight. In other embodiments, it is envisaged that doses may be in the range of about 0.5 μg compound/kg body weight to about 20 μg compound/kg body weight. In other embodiments, the doses may be about 0.5, 1, 3, 6, 10, or 20 mg/kg body weight. Of course, this dosage amount may be adjusted upward or downward, as is routinely done in such treatment protocols, depending on the results of the initial clinical trials and the needs of a particular patient.

In some cases, the immune checkpoint inhibitor, e.g., the inhibitor of PDL-1 or PD-1, is administered at a dosage of 0.01-10 mg/kg (e.g., 0.01, 0.05, 0.1, 0.5, 1, 5, or 10 mg/kg) bodyweight. For example, the PDL-1 or PD-1 inhibitor is administered in an amount of 0.01-30 mg (e.g., 0.01, 0.05, 0.1, 0.5, 1, 5, 10, 20, or 30 mg) per dose. In another example, the immune checkpoint inhibitor, e.g., the anti-PD-L1 or anti-PD-1 antibody, is administered in the dose range of 0.1 mg/kg to 10 mg/kg of body weight. In some cases, the XL313 antibody or the HU177 antibody is administered at a dosage of 0.01-10 mg/kg (e.g., 0.01, 0.05, 0.1, 0.5, 1, 5, or 10 mg/kg) bodyweight. For example, the XL313 antibody or the HU177 antibody is administered in an amount of 0.01-30 mg (e.g., 0.01, 0.05, 0.1, 0.5, 1, 5, 10, 20, or 30 mg) per dose. For example, the dose range of Mab XL313 or Mab HU177 is from 0.1 mg/kg to 25 mg/kg of body weight.

The compositions of the invention (e.g., inhibitor of PDL-1 or PD-1, XL313 antibody, and HU177 antibody) are administered once per month, twice per month (i.e., every two weeks), every week, once per day, twice per day, every 12 hours, every 8 hours, every 4 hours, every 2 hours or every hour. The compositions of the invention (e.g., inhibitor of PDL-1 or PD-1, XL313 antibody, and HU177 antibody) are administered for a duration of 1 day, 2 days, 3 days, 4 days, 5 days, 6 days, 7 days, 2 weeks, 3 weeks, 4 weeks, five weeks, six weeks, 2 months, 3 months, 4 months, 5 months, 6 months, 7 months, 8 months, 9 months, 10 months, 11 months, 12 months, 2 years, 3 years, 4 years, 5 years or more. For example, the composition of the invention (e.g., inhibitor of PD-L1, XL313 antibody, and HU177 antibody) are administered one dose every two weeks for 4 to 6 weeks or until the disease is treated.

Also provided is a method of treating a disease characterized by abnormal immune suppression in a subject by identifying a subject, e.g., a human, that has been diagnosed with a disease characterized by abnormal immune suppression, administering an immune checkpoint inhibitor, and administering an antagonist of an integrin, thereby treating in the subject.

Suitable immune checkpoint inhibitors comprise an inhibitor of CTLA-4, PD-1, PDL-1, Lag3, LAIR1, or LAIR 2. For example, the immune checkpoint inhibitor comprises a CTLA-4 antibody, a PD-1 antibody, a PDL-1 antibody, a Lag3 antibody, a LAIR1 antibody, or a LAIR 2 antibody.

Preferably, the integrin comprises integrin αvβ3. For example, the antagonist of integrin αvβ3 comprises an antibody targeting αvβ3 binding RGDKGE (SEQ ID NO: 1) containing collagen epitope. Alternatively, the integrin comprises integrin α10β1. For example, the antagonist of integrin α10β1 comprises an antibody targeting α10β1 binding CPGFPGFC (SEQ ID NO: 16)-containing collagen epitope.

The methods described herein can be used in conjunction with one or more chemotherapeutic or anti-neoplastic agents. In some cases, the additional chemotherapeutic agent is radiotherapy. In some cases, the chemotherapeutic agent is a cell death-inducing agent.

Suitable diseases characterized by abnormal immune suppression include Type I diabetes, lupus, psoriasis, scleroderma, hemolytic anemia, vasculitis, Graves' disease, rheumatoid arthritis, multiple sclerosis, Hashimoto's thyroiditis, Myasthenia gravis, and vasculitis.

In some cases, the immune checkpoint inhibitor (e.g., CTLA-4 antibody, a PD-1 antibody, a PDL-1 antibody, Lag3, LAIR1, or LAIR 2) is administered at a dosage of 0.01-10 mg/kg (e.g., 0.01, 0.05, 0.1, 0.5, 1, 5, or 10 mg/kg) bodyweight. For example, the PDL-1 or PD-1 inhibitor is administered in an amount of 0.01-30 mg (e.g., 0.01, 0.05, 0.1, 0.5, 1, 5, 10, 20, or 30 mg) per dose. In another example, the antibody is administered in the dose range of 0.1 mg/kg to 10 mg/kg of body weight. In some cases, the antagonist of integrin αvβ3 is administered at a dosage of 0.01-10 mg/kg (e.g., 0.01, 0.05, 0.1, 0.5, 1, 5, or 10 mg/kg) bodyweight. In some cases, the XL313 antibody or the HU177 antibody is administered in an amount of 0.01-30 mg (e.g., 0.01, 0.05, 0.1, 0.5, 1, 5, 10, 20, or 30 mg) per dose.

The compositions of the invention (e.g., immune checkpoint inhibitor and antagonist of integrin αvβ3) are administered once per month, twice per month (once every two weeks), once a week, once per day, twice per day, every 12 hours, every 8 hours, every 4 hours, every 2 hours or every hour. The compositions of the invention (e.g., inhibitor of PDL-1 or PD-1, XL313 antibody, and HU177 antibody) are administered for a duration of 1 day, 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 2 years, 3 years, 4 years, 5 years or more. The composition of the invention (e.g., inhibitor of PDL-1 or PD-1, XL313 antibody, and HU177 antibody) are administered one dose every two weeks for 4 to 6 weeks or until the disease is treated.

Also provided is a method of treating a disease characterized by an overactive immune response (e.g., an autoimmune disease) in a subject, e.g., a human subject, that has been diagnosed with an overactive immune response by administering a peptide comprising collagen or a fragment thereof, thereby treating overactive immune response in the subject. Suitable types of collagen include collagen type-I, collagen type II, collagen type III, and collagen type-IV (e.g., the alpha 6 chain of collagen type-IV). For example, the peptide comprises RGDKGE (SEQ ID NO: 1) or CPGFPGFC (SEQ ID NO: 16).

In some cases, the autoimmune disease comprises Graves' disease, Hashimoto's thyroiditis, Systemic lupus erythematosus (lupus), Type 1 diabetes, multiple sclerosis or rheumatoid arthritis.

Methods for healing a wound in a subject, e.g., a human subject with a wound, are carried out by administering a peptide comprising collagen or a fragment thereof to the wound of the subject, thereby healing a wound in the subject. For example, the peptide is administered to a site that is about 0.1 mm, 0.5 mm, 1 mm, 2.5 mm, 5 mm, 10 mm, 15 mm, 20 mm, or 40 mm away from a perimeter or margin of the wound. Alternatively, the peptide is administered directly to the wound itself.

Suitable types of collagen include collagen type-I, collagen type II, collagen type III, and collagen type-IV (e.g., the alpha 6 chain of collagen type-IV). For example, the peptide comprises RGDKGE (SEQ ID NO: 1) or CPGFPGFC (SEQ ID NO: 16).

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

Adoptive cell therapy (ACT) is an immune cellular therapy that involves using isolated tumor-infiltrating T-cells (TILs) or genetically engineered T-cells that are infused into a subject to treat diseases such as cancer (1-3). ACT is based in part on the idea that specific subsets of effector T-cells directed to tumor antigens can be expanded ex-vivo and then re-infused into patients in hopes that these cells will rapidly localize to the malignant lesion and efficiently kill the malignant tumor mass. A central caveat of this therapeutic approach is that the T-cells have an efficient mechanism that allows the cells that have been injected into the circulation, to migrate out of tumor associated blood vessels in order to gain access to the tumor (1-3).

It is well accepted that the vascular endothelium of malignant tumors is very different than that of normal blood vessels (4-6). In general, tumor vessels exhibit a higher level of cellular proliferation, they are also often characterized as being unstructured, leaky, exhibiting disrupted, often incompletely formed vascular basement membrane networks that underlay the endothelial cells (4-6). Vascular basement membranes (BMs) are predominately composed of different isoforms of extracellular matrix (ECM) proteins such as collagen type-IV and laminin that form a network of sheet-like structures that provide a restrictive vascular ECM barrier (4-6). Importantly, tumor blood vessels provide a major conduit for a variety of innate immune system cells such as macrophages as well as a variety of different adaptive immune cells, including various subsets of T-cells. Thus, the tumor-associated vasculature represents a critical conduit through which many cell types migrate into and out of the tumor microenvironment in a bi-directional manner. In this regard, the mechanisms that regulate the ability of immune cells to traverse the interconnected ECM barrier of the tumor is not completely understood. While much progress has been made in understanding the molecular mechanism of ameboid cell motility that controls trans-endothelial migration through the endothelial cell layer, much less is known about the mechanisms that control the ability of immune cells to breach and migrate through the restrictive subendothelial basement membrane.

Great interest has emerged in the field of Adoptive Cell Therapy (ACT) as a potential new strategy for the treatment of malignant tumors (1-3). However, one critical caveat that is required for this strategy to be a successful, is that the T-cells that are being re-introduced back into the subject's circulation, must efficiently gain access to the tumor site, by leaving the circulation and migrating through the restrictive subendothelial basement membrane (1-3). The ability of T-cells to migrate through the basement membrane is critical to all forms of ACT, including strategies using isolated and expanded tumor-infiltrating lymphocytes (TILs), engineered T-cells with high affinity T-cell receptors, and chimeric antigen receptor (CAR) T-cells (1-3). While ACT has been used clinically with some success, its activity has been largely restricted to hematological tumors (1-3). In fact, the different forms of ACT have demonstrated only modest activity in some tumor types such as melanomas, and have also been associated with significant side effects associated with T-cell infiltration into normal non-diseased organs. Thus, focused efforts are now underway to improve the specificity and activity of ACT by enhancing the T-cell survival, improving their cell killing ability, reducing their exhausted phenotype, and improving their ability to accumulate in sufficient numbers at the tumor sites to avoid off target effects.

Angiogenesis, the process by which new blood vessels form from pre-existing vessels plays a critical role in normal and pathological events. Efforts are underway to more precisely define the interconnected mechanisms that control this crucial biological process in order to develop more effective strategies to control neovascular diseases. Significant advances have been made in identifying molecular regulators of angiogenesis and their associated signaling pathways. A more precise understanding of angiogenic signaling pathways and the networks of regulatory feedback loops operating within distinct cellular compartments has provided important clues to help explain the modest clinical impact of many anti-angiogenic strategies. For example, while vascular endothelial growth factor (VEGF) induces pro-angiogenic signaling leading to enhanced endothelial cell migration, growth, and survival, VEGF-induced blood vessels are often characterized as immature, unstable, and leaky and can regress in the absence of additional signaling events. VEGF stimulation under specific circumstances may lead to inhibition of angiogenesis in the context of altered PDGF signaling due to disruption of pericyte recruitment. These unexpected findings provide evidence of a negative role for the VEGF/VEGFR signaling during new vessel development. Similarly, studies have provided evidence for both a positive and a negative role for integrin αvβ3 in angiogenesis.

A wide array of alterations in the composition and biomechanical properties of extracellular matrix (ECM) proteins are known to occur during angiogenesis and studies are beginning to define how these changes contribute to new blood vessel development. Among the key cell surface molecules that play roles in mechano-transduction to facilitate information flow from outside the cell to the inside are integrin receptors. Integrins may act like information hubs by sensing diverse extracellular inputs and relaying this information into a complex network of intracellular circuits that ultimately modulate cellular behavior. The precise molecular mechanisms by which cells fine-tune their response to changes within the stromal microenvironment are not completely understood. Further complicating the understanding of new vessel development is the expanding number of cell types that contribute to tissue specific control of angiogenesis such as distinct subsets of stromal fibroblasts, progenitor cells and a variety of inflammatory cells such as neutrophils, mast cells and macrophages. The roles played by these diverse cells during angiogenesis range from secretion of cytokines, chemokines and proteolytic enzymes to the differential expression of other pro and anti-angiogenic factors. Thus, tight control mechanisms must operate to allow coordination between these diverse compartments to govern tissue specific vascular responses.

Integrins are molecules with the ability to detect compositional and structural changes within the ECM and integrate this information into a network of signaling circuits that coordinate context dependent cell behavior. Among the most well studied integrins known to play a role in angiogenesis is αvβ3. The complexity by which αvβ3 regulates angiogenesis is illustrated by the fact that this receptor may exhibit both pro and anti-angiogenic functions. It is indicated that the distinct biological responses stimulated by binding to αvβ3 may depend on many factors including the mechanical and biochemical features of the particular ligands, the cell types within which αvβ3 is expressed as well as the concentration and manner by which the ligands are presented to the receptor. For example, studies indicate that αvβ3 binding to specific NC1 domains of collagen or selected RGD peptides can induce apoptosis, induce arteriole contraction and inhibit angiogenesis while other αvβ3 ligands may promote cell survival, induce vascular dilation and support angiogenesis. These observations are consistent with the notion that the final outcome of αvβ3-mediated signaling may depend to a large extent on the particular characteristics of the ligand. While a wealth of data has shown that RGD peptides can inhibit angiogenesis when administered exogenously, the approach of using a cyclic RGD peptide to control tumor growth failed to significantly impact glioblastoma progression and patient survival in late stage clinical testing. Interestingly, studies have indicated that specific RGD peptides may activate β3 integrins and under defined experimental conditions induce angiogenesis and tumor growth. These findings and other studies suggesting that amino acids C-terminal to the RGD motif play roles in governing integrin selective binding, prompted the examination of the biological significance of naturally occurring RGD containing epitopes on angiogenesis.

For example, multiple pro-angiogenic roles have been proposed for αvβ3 as cyclic arginine-glycine-aspartic acid (RGD) containing peptides and antibodies targeting this integrin inhibit angiogenesis in animal models. In contrast, enhanced angiogenesis was detected in tumors growing in αvβ3 null mice. Interestingly, reduced pathological angiogenesis was detected in transgenic mice expressing signaling deficient 3 integrin, which resulted in part from defective recruitment of bone marrow derived cells rather than specific endothelial cell defects. Moreover, evidence suggests that 3 integrin may play a more prominent role in the early stages of angiogenesis when new vessels begin to form, as reduction in endothelial cell expression of β3 integrin impaired early stage pathological angiogenesis, but had little effect on later maturation stages once vessels had formed. These studies, together with many others suggests αvβ3-mediated regulation of angiogenesis is complex, temporally regulated and is not solely dependent on adhesive events, but also involves downstream signaling, the consequences of which may depend on the cell type and composition of the local extracellular microenvironment. Because of the opposing biological responses observed following modulation of some angiogenic regulatory molecules, it is not surprising that anti-angiogenic strategies based on targeting these factors have met with limited clinical success.

Given the importance of integrin-extracellular matrix (ECM) interactions in modulating the intensity and specificity of growth factor signaling, it is important to define how diverse components within the local vascular microenvironment function cooperatively to regulate angiogenesis. Interestingly, distinct αvβ3 ligands may stimulate opposing biological outcomes. For example, certain NC1 domains of collagen may bind αvβ3 and induce proapoptotic responses while binding of other αvβ3 ligands may promote cell growth and survival (19-22). Given these findings and the complex biological effects observed following direct targeting of αvβ, an alternative therapeutic approach to control signaling from αvβ3 might involve specific targeting of the pro-angiogenic ligands of αvβ3 rather than directly targeting the receptor itself. Proteolytic remodeling of the ECM can generate integrin binding cryptic epitopes that play functional roles in angiogenesis including the LPG×PG containing HU177 cryptic epitope present in multiple types of collagen and the HUIV26 cryptic epitope, which is present in collagen type-IV. While the HUIV26 epitope is recognized by αvβ, it is not specifically composed of an RGD motif.

Sequence analysis of RGD sites within collagen type-I indicate that the KGE tri-peptide motif that is C-terminal to the RGD site was highly conserved among diverse species, while considerable variation is observed in the other collagen RGD flanking sequences. While all five of the collagen RGD epitopes can support cell binding, the highly conserved RGDKGE (SEQ ID NO: 1) collagen peptide P-2 may play a functional role angiogenesis and inflammation given that Mab XL313 directed to this epitope, but not an antibody that recognizes the other three RGD collagen sites, inhibited angiogenesis and inflammation in vivo. While the precise difference between the three other naturally occurring non-RGDKGE (SEQ ID NO: 1) containing collagen epitopes is not clear, one or more of these epitopes were detected in vivo in addition to the RGDKGE (SEQ ID NO: 1) epitope. Given that these RGD containing epitopes are thought to be largely cryptic and not readily accessible to cell surface receptor, the findings are consistent with active collagen remodeling resulting in generation neoepitopes during new vessel formation.

Because of the importance of RGD sequences in mediating some integrin-dependent interactions and the roles of amino acids flanking the core RGD motif in establishing integrin-binding specificity and affinity, the ability of RGD motifs within collagen differentially regulate angiogenesis was determined. Sequence analysis of collagen type-I revealed that five different cryptic RGD motifs are present, each with unique flanking sequences. Surprisingly, the C-terminal KGE flanking sequence of one of these RGD motifs is highly conserved in species as diverse as Xenopus and man. In contrast, significant sequence and positional variation exists within the other flanking sequences among different species.

ECM remodeling occurs as an early event during angiogenesis and multiple proteolytic enzymes including matrix metalloproteinase (MMPs) as well as serine and cysteine proteases the have the capacity to degrade intact or structurally altered forms of collagen (18, 58). While the in vitro studies indicate that MMP-2-mediated degradation of collagen can lead to the generation of low molecular weights fragments recognized by Mab XL313, the precise mechanism by which the RGDKGE (SEQ ID NO: 1) collagen epitope is generated in vivo is not completely understood. Analysis of angiogenic Chorioallantoic Membrane (CAM) tissues suggests that a subset of macrophages may be an important source of the RGDKGE (SEQ ID NO: 1) epitope. Activated macrophages with M2-like characteristics can express multiple enzymes capable of degrading collagen and in turn can internalize and further degrade collagen into small low molecular weight fragments (35, 38). While little evidence exist that macrophages generate and deposit intact triple helical collagen type-I, studies have indicated that certain isoforms of collagen may be expressed (59). Consistent with previous reports, intact collagen was detected; however low molecular weight RGDKGE (SEQ ID NO: 1) containing collagen fragments in both whole cell lysates and serum free conditioned medium from macrophages like cell lines was detected. While the studies do not rule out macrophage mediated collagen internalization as a contributing factor to the in vivo generation the RGDKGE (SEQ ID NO: 1) collagen epitope, the in vitro studies were carried out in the absence of serum or exogenously added collagen, and thus are consistent with the active generation of the RGDKGE (SEQ ID NO: 1) collagen fragment by macrophages.

Activated macrophages including M2-polarized macrophages have been implicated in supporting angiogenesis and inflammation as multiple factors secreted by these cells exhibit pro-angiogenic activities. While many studies indicate that synthetic RGD containing peptides inhibit angiogenesis and tumor growth, the findings provide the first evidence that macrophages may generate and release an RGDKGE (SEQ ID NO: 1) containing collagen epitope that may exhibit pro-angiogenic activity. Importantly, previous studies have suggested that certain RGD-peptides can activate αvβ3 and may enhance vascular permeability, which might lead to release of inflammatory factors, which may in turn contribute to the formation of new blood vessels.

To examine possible mechanisms by which the RGDKGE (SEQ ID NO: 1) collagen peptide might regulate angiogenesis, endothelial cell receptors for this motif were identified. While the possibility that additional non-integrin receptors may bind this collagen epitope is not ruled out, the data suggest that αvβ3 can function as an endothelial cell receptor for the RGDKGE (SEQ ID NO: 1) motif Interestingly, αvβ3 bound both the RGDKGE (SEQ ID NO: 1) and RGDAPG (SEQ ID NO: 11) collagen peptides, yet only RGDKGE (SEQ ID NO: 1) peptide significantly induced angiogenesis and inflammation in vivo. These findings are consistent with the notion that distinct RGD containing αvβ3 ligands may promote different biological responses. Signaling downstream from αvβ3 is complex and studies have indicated that simple binding of β3 integrin does not necessarily lead to productive outside-in integrin signaling (61). In fact, the capacity of 33 integrins to promote outside-in signaling depends on multiple factors including the extent of receptor clustering and subsequent generation of mechanical tension within the actin cytoskeleton, recruitment of adaptor and accessory proteins such as Gal3, and Kindlin-2 and the association of the integrin with protein tyrosine phosphatases and certain growth factor receptors. While the exact mechanisms leading to RGDKGE (SEQ ID NO: 1)-mediated αvβ3 signaling is not completely understood, endothelial cell interactions with the RGDKGE (SEQ ID NO: 1) peptide in the absence of serum led to enhanced phosphorylation of 33 integrin on tyrosine 747 and of Src phosphorylation at tyrosine 416. These data and others are consistent with an early mechanical mediated activation of β3 integrin that depends on Src given that blocking Src activity reduced 33 phosphorylation following binding to the RGDKGE (SEQ ID NO: 1) motif.

Integrin signaling and Src activation are known to regulate the architecture of the actin cytoskeleton. Moreover, Src family kinases regulate P38MAPK, and activation of P38MAPK is thought to enhance actin stress fiber formation in endothelial cells and regulate angiogenesis in vivo. The findings provide insight into the coordinated roles of P38MAPK and Src in regulating RGD-dependent endothelial cell signaling through αvβ3 as interactions with the RGDKGE (SEQ ID NO: 1) cryptic collagen epitope leads to enhanced P38MAPK phosphorylation in a Src-dependent manner. Moreover, RGDKGE (SEQ ID NO: 1)-induced angiogenesis in vivo was associated with enhanced levels of phosphorylated P38MAPK, and this angiogenic response was reduced by an inhibitor of P38MAPK. These findings are consistent with the notion that RGDKGE (SEQ ID NO: 1) stimulated angiogenesis depends on P38MAPK.

Recent studies have suggested a role for actin stress fibers and mechanical tension in promoting nuclear accumulation of Yes-associated protein (YAP), where it is thought to function in conjunction with TEA domain transcription factor (TEAD) transcription factors (e.g., TEAD1 (TEF-1/NTEF), TEAD2 (TEF-4/ETF), TEAD3 (TEF-5/ETFR-1), and TEAD4 (TEF-3/ETFR-2/FR-19)) in regulating gene expression (46-50). Given data suggesting a role for YAP in regulating endothelial cell growth and angiogenesis, the subcellular distribution of YAP in endothelial cells following interaction with the RGDKGE (SEQ ID NO: 1) collagen peptide was examined. The data indicate that endothelial cell interaction with the RGDKGE (SEQ ID NO: 1) epitope was associated with enhanced nuclear accumulation of YAP. Integrin signaling may play a role in the regulation of YAP as studies have implicated a role for β1 integrins expressed in skeletal stem cells and ay integrins expressed in osteoblasts in governing YAP subcellular localization. The findings are consistent with a mechanism by which αvβ3-mediated binding to the RGDKGE (SEQ ID NO: 1) epitope, but not the related RGDAPG (SEQ ID NO: 11) epitope stimulates a signaling cascade leading to enhanced nuclear accumulation of YAP that depends on Src and/or P38MAPK. This possibility is supported by the findings that reduced levels of nuclear YAP was detected following αvβ3-mediated interaction with RGDKGE (SEQ ID NO: 1) peptide in endothelial cells in which Src or P38MAPK was inhibited. Given the documented role of YAP in governing cell growth coupled with the ability of the RGDKGE (SEQ ID NO: 1) collagen peptide to stimulate nuclear accumulation of YAP and enhance endothelial cell growth, it is possible that the RGDKGE (SEQ ID NO: 1) collagen peptide-induced endothelial cell growth is YAP dependent. Consistent with this possibility, no enhancement of endothelial cell growth was detected following knockdown of YAP in endothelial cells stimulated with the RGDKGE (SEQ ID NO: 1) collagen peptide, even though these cells are capable of proliferating as stimulation with VEGF or high levels of serum enhanced their growth. Given the studies, it is possible that part of the FGF-2 induced angiogenic response observed in the chick CAM model might involve the recruitment of macrophages that generate a previously uncharacterized RGDKGE (SEQ ID NO: 1) containing cryptic collagen epitope that binds to αvβ3 leading to Src and P38MAPK activation and nuclear accumulation of YAP. Given that YAP is known to regulate a wide array of genes that may impact angiogenesis and inflammation including CTGF and Cry61, it is likely that the RGDKGE (SEQ ID NO: 1) collagen epitope may initiate a complex pro-angiogenic program in vivo involving YAP-associated regulation of multiple pro-angiogenic molecules and not simply be restricted to only enhancing endothelial cell growth.

Collectively, these results provide evidence that a highly conserved RGDKGE (SEQ ID NO: 1) containing collagen epitope can be generated by a subset of macrophages and the RGDKGE (SEQ ID NO: 1) collagen epitope can stimulate pro-inflammatory and angiogenic activity. Binding of the RGDKGE (SEQ ID NO: 1) collagen epitope to β3 integrin can initiate a signaling pathway in endothelial cells leading to activation of Src and P38MAPK ultimately leading to nuclear accumulation of YAP and enhance cell growth. The results presented herein provide cellular and molecular insight into how an endogenously generated RGD containing cryptic collagen epitope may promote rather that inhibit angiogenesis. Given the complexity of αvβ3 functions and the growing body of evidence that the final outcome of αvβ3 binding may depend on the nature of the particular ligand, the findings provide support for an alternative strategy to help control the biological activity of β3 integrin by specific targeting of endogenous pro-angiogenic ligands of αvβ3 rather than direct targeting of the receptor itself.

Integrin αvβ3 plays a functional role in promoting immune suppression in part by upregulating the expression of the immune checkpoint regulatory protein PDL-1. Thus, targeting αvβ3 with function blocking (signal blocking) antagonists of αvβ3 or reducing expression of αvβ3 led to reduced expression of PDL-1. Thus antagonist of αvβ3 may enhance the anti-tumor efficacy of immune checkpoint therapy. Importantly, while immune check point inhibitors are known to provide some anti-tumor activity in humans, this partial anti-tumor activity is only observed in a fraction of treated subjects. Previously, the identification of compounds and combination treatment strategies to enhance the efficacy of immune checkpoint inhibitors such as CTLA-4, PDL-1 and PD-1 antibodies was described (see, e.g., US Patent Publications 2017-0065716 (Granted as U.S. Pat. No. 10,881,732), and 2017-0240638 (Granted as U.S. Pat. No. 10,906,977), which are incorporated herein by reference).

Therapeutic blockade of immune checkpoint regulatory molecules such as CTLA-4 and PD-1/PDL-1 signaling is known to be associated with significant immune related side effects including inflammation. Given these known side effects of immune checkpoint therapy and the ability of specific ligands of αvβ3 integrin such as the RGDKGE (SEQ ID NO: 1) containing collagen epitope to potentially induce inflammation in vivo (and the anti-stromal cell migratory activity of Mab HU177), combining antagonist of αvβ3 (or an antagonist of HU177 or an antagonist of α10β1) with anti-PD-1/PDL-1 antagonists may reduce the inflammatory side effects associated with immune checkpoint inhibitor therapy.

Previously described in vivo animal data indicated that melanoma tumors that express integrin αvβ3 express the immune checkpoint protein PDL-1, while the same tumor cell type that was selected for lack of functional αvβ3 exhibited little detectable PDL-1. Second, cellular interactions of tumor cells as well as endothelial cells with ECM proteins (denatured collagen and the RGDKGE (SEQ ID NO: 1) collagen epitope) that documented ligands of integrin αvβ3 lead to upregulated expression of PDL-1. Third, a function blocking antibody directed specifically to integrin αvβ3 reduced expression of PDL-1 in tumor cells. Finally, an antibody (Mab XL313) that specifically blocks the binding of an RGDKGE (SEQ ID NO: 1) epitope to αvβ3 and inhibits downstream signaling from αvβ3 enhanced the anti-tumor efficacy of an immune checkpoint inhibitor (anti-PDL-1) in vivo.

Previous studies have indicated that blockade of immune checkpoint proteins such as PDL-1, PD-1, and LAG-3 can result in some anti-tumor activity. These anti-tumor effects, however were only partial and only occurred in a fraction of the treated subjects. Previously, the identification of compounds that enhance the effect of immune checkpoint inhibitors such as antibodies targeting CTLA-4, PD-1 and/or PDL-1 was described. To this end, studies have suggested that combining immune checkpoint inhibitors with other chemotherapy drugs may enhance the anti-tumor activity. Importantly, a common side effect that can limit the use of immune checkpoint inhibitors is the active induction of inflammatory conditions such as dermatitis, pneumonitis and colitis. Part of the inflammatory process in vivo may involve alterations in expression of inflammatory cytokines and infiltration of activated stromal cells such as activated fibroblasts. In this regard, as described in detail previously, antagonists of the XL313 epitope (Mab XL313) not only enhance the therapeutic activity of an anti-PDL-1 antibody therapy in a mouse model, but Mab XL313 epitope potently inhibits inflammation in vivo. Antagonists of the HU177 epitope (Mab HU177) not only enhance the therapeutic activity of an anti-PDL-1 antibody therapy in a mouse model, but Mab HU177 inhibits activated fibroblast migration and accumulation within tumors in vivo. Additionally a αvβ3 binding RGDKGE (SEQ ID NO: 1) containing collagen epitope that stimulates αvβ3 integrin signaling was shown to significantly enhance inflammation in vivo. Given these findings, it is possible that blocking αvβ3 signaling with an antagonist of αvβ3 would not only enhance the therapeutic activity of an anti-PDL-1/PD-1 or CTLA-4 based therapy, but may also potently inhibit inflammation in vivo. These findings are consistent with the notion that combining an antagonist of XL313 epitope (or antagonist of αvβ3) or an antagonist of HU177 epitope (or antagonist of α10β1) with immune checkpoint inhibitor therapy not only enhances its efficacy, but also reduces the inflammatory side effects observed with the immune checkpoint inhibitor therapy.

Evidence that an RGDKGE (SEQ ID NO: 1) containing cryptic collagen epitope is generated by a subset of macrophages and this motif promoted rather than inhibited angiogenesis was previously described. These findings are surprising given the wealth of experimental data indicating the high concentration of RGD peptides inhibit rather than induce angiogenesis. Increasing evidence suggests that low concentrations of certain RGD peptides may actually enhance angiogenesis and tumor growth, which may explain at least in part the minimal impact of cyclic RGD peptide antagonists of αvβ3 and αvβ5 in human clinical trials. In addition to variations in concentrations that alter the biological response of certain RGD peptides, the specific composition of the amino acids C-terminal to RGD motif within naturally occurring epitopes may confer unique pro-angiogenic and inflammatory activity. Taken together, these results are consistent with a mechanism by which the RGDKGE (SEQ ID NO: 1) collagen epitopes induce angiogenesis and inflammation by stimulating mechanical activation of αvβ3 leading to Src-dependent phosphorylation of P38 MAPKinase that promotes nuclear accumulation of the Yes-associated protein (YAP) and enhanced endothelial cell growth.

### Cryptic Collagen Epitopes

Studies have documented the capacity of extracellular matrix (ECM) proteins containing the short amino acid sequence RGD to support interactions mediated by integrin receptors (33). The ability of cells to interact with RGD sites within the context of larger glycoproteins depends on many factors, some of which include the adjacent flanking sequences surrounding the core RGD tri-peptide as well as the geometrical configuration of the intact molecule and how these molecules are oriented within the context of the interconnected network of other ECM proteins. Flanking sequences immediately C-terminal to the RGD site can govern integrin selective binding. RGD motifs can be cryptic and inaccessible to cell surface receptors as is illustrated in the case of triple helical collagen (34). In this regard, five different cryptic RGD containing sites exist within human collagen type-I, each with distinct flanking sequences (Table 1).

TABLE 1
Cryptic epitopes of collagen

| | AA sequence | |
|---|---|---|
| Peptide | (SEQ ID NO:) | Location |
| | RGDKGE | |
| | (SEQ ID NO: 1) | |
| P-1 | KGDRGDAPG | Colla1 742-750 |
| | (SEQ ID NO: 2) | |
| P-2 | QGPRGDKGE | Colla1 1090-1098 |
| | (SEQ ID NO: 3) | |
| P-3 | AGSRGDGGP | Colla2 774-782 |
| | (SEQ ID NO: 12) | |
| P-4 | QG1RGDKGE | Colla2 1002-1010 |
| | (SEQ ID NO: 13) | |
| P-5 | RGPRGDQGP | Colla2 819-827 |
| | (SEQ ID NO: 14) | |
| P-C | QGPSGAPGE | NA |
| | (SEQ ID NO: 15) | |
| | CPGFPGFC | |
| | (SEQ ID NO: 16) | |
| CP | CQGPGGAAGGC | NA |
| | (SEQ ID NO: 4) | |

Five different cryptic RGD containing sites exist within human collagen type-I, each with distinct flanking sequences. Synthetic peptides of these five sequences were generated and designated P-1 through P-5 as shown above. Additionally, control peptides (P-C and CP) were generated lacking the RGD tri-peptide motif. The sequences in Table 1 correspond to the following SEQ ID NOs.—P-1: KGDRGDAPG (SEQ ID NO: 2), P-2: QGPRGDKGE (SEQ ID NO: 3), P-3: AGSRGDGGP (SEQ ID NO: 12), P-4: QGIRGDKGE (SEQ ID NO: 13); P-5: RGPRGDQGP (SEQ ID NO: 14); P-C: QGPSGAPGE (SEQ ID NO: 15), and CP: QGPGGAAGGC (SEQ ID NO: 4).

Cryptic CPGFPGFC (SEQ ID NO: 16)-containing collagen epitope is HU177 epitope. Cryptic RGDKGE (SEQ ID NO: 1)-containing collagen epitope is XL313 epitope.

### Examples

### Reagents, Kits, Chemicals and Antibodies

Ethanol, methanol, acetone, bovine serum albumin (BSA), crystal violet, phosphate-buffered saline (PBS), purified human collagen type-IV and collagen type-I, AMPA, 3,3,5,5′ tetramethybenzidine (TMB), phosphatase inhibitor cocktail, and cortisone acetate (CA) were from Sigma (St Louis, Mo.). MMP2 was from Chemicon/Millipore (Billerica, Mass.). FBS was from Science Cell (Carlsbad, Calif.). Fibroblast growth factor-2 (FGF-2) was obtained from R&D Systems (Minneapolis, Minn.). Nuclear/Cytoplasmic fractionation kit was from Thermo Scientific (Waltham, Mass.). P38MAPK inhibitor, SB202190 was obtained from Cal-Boichem (San Diego, Calif.). RIPA buffer, protease inhibitor, and Src inhibitor (PP2) were from Santa Cruz (Santa Cruz, Calif.). Anti-vWf antibody was from BD Pharmingen (San Diego, Calif.). Antibodies directed to P38MAPK, phospho-P38MAPK (Thr-180/Tyr-182), Src, and phospho-Src (Tyr 416), were from Cell Signaling Technology (Danvers, Mass.). Antibodies against tubulin, total binding protein (TBP), YAP, P3, and phospho-133 (Tyr747) were from Santa Cruz (Santa Cruz, Calif.). Anti-Igfbp4 and anti-MMP9 antibodies were obtained from Abcam (Cambridge, Mass.). Function blocking antibodies P4C10 (anti-(31), LM609 (anti-av (33) and P1F6 (anti-av (35) were from R&D Systems (Minneapolis, Minn.). HRP-conjugated secondary antibodies were from Promega (Madison, Wis.). Anti-collagen type-I antibody was from Rockland (Limerick, Pa.) and anti-collagen type-IV was from Millipore (Billerica, Mass.). Mouse monoclonal antibodies XL313, and XL166 were developed. Alexa-488, Alexa-594, streptavidin Alexa-594, and phalloidin Alexa-594 labeled antibodies were from Invitrogen (Carlsbad, Calif.). Synthetic collagen RGD containing peptides (P-1; CKGDRGDAPGC (SEQ ID NO: 5), P-2; CQGPRGDKGEC (SEQ ID NO: 6), P-3; CAG-SRGDGGPC (SEQ ID NO: 7), P-4; CQGIRGDKGE (SEQ ID NO: 8), P-5; CRGPRGDQGPC (SEQ ID NO: 9) and peptide control P-C; CQGPSGAPGEC; (SEQ ID NO: 10) were obtained from QED Biosciences (San Diego, Calif.).

### Western Blots

Whole cell and CAM tissue lysates were collected in RIPA buffer (10 mM Tris-HCl, pH 8.0; 1 mM EDTA; 0.5 mM EGTA; 1% Triton X-100; 0.1% Sodium Deoxycholate; 0.1% SDS; 140 mM NaCl) supplemented with 1× protease inhibitor and 1× phosphatase inhibitor and were run on polyacrylamide gels using denaturing conditions. Prior to loading the gels, 6× sample buffer was added to each of the lysates (final concentration 1×) and boiled for five minutes. Twenty to 50 ng of total protein were loaded into each lane. For detection of proteins larger than 50 kD, 10% gels were used; for proteins smaller than 50 kD 15% gels were used. Gels were run at 60 volts (V) until the dye front passed through the stacking gel, and then the voltage was increased to 100 V for the remainder of the separation. Precision Plus protein standards (Bio-Rad) were used to visualize migration. Protein was transferred to nitrocellulose membranes using a wet tank system and blocked for 1 hour using 10% non-fat dried milk in tris-buffered saline with 0.01% Tween-20 (TBS-T). Membranes were incubated with primary antibodies (anti-coll-I (1:250), anti-coll-IV (1:250), Mab XL313 (2 μg/ml), Mab XL166 (2 μg/ml) anti-13-actin (1:5000), anti-phospho-133 (1:7000), anti-133 (1:1000), anti-phospho-Src (1:500), anti-Src (1:500), anti-phospho-P38MAPK (1:500), anti P38MAPK (1:2000), anti-YAP (1:500), anti-Tubulin (1:2000), anti-TBP (1:1000)) in 5% BSA in TBS-T overnight at 4° C. with gentle agitation. Membranes were washed 3 times in TBS-T for five minutes. Blots were then incubated with HRP conjugated secondary antibodies (1:15000) in 1% non-fat milk in TBS-T for 1 h. Membranes were washed a second time as indicated above and exposed to chemiluminescent substrate for three minutes prior to exposure to autoradiography film in a dark room. Western blot bands were quantified using Image J software based on pixel intensity.

### Example 1: Selective Expression of Proteolyzed and Denatured Forms of Collagen Associated with Tumor Vasculature

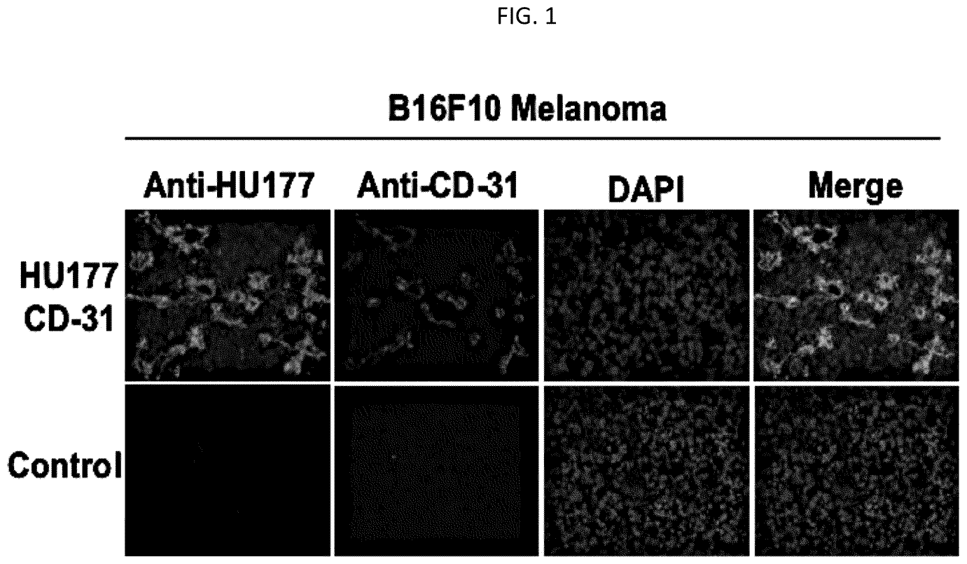

Given our previous studies and those of other investigators which have demonstrated the selective expression of denatured and proteolyzed forms of collagen within tumor vessels (5-10), we began studying the migratory behavior of T-cells on denatured collagen type-IV. First, we confirmed the expression of denatured forms of collagen is associated with tumor blood vessels. We co-stained B16F10 tumors with antibodies directed to a marker of blood vessels (CD31) as well as using the anti-HU177 antibody which is selectively directed to denatured forms of collagen (7-9). As shown in FIG. 1, denatured collagen (green) was associated with blood vessels (red) in these tumors. A B16F10 tumor sample, stained with a secondary antibody only, was used as a negative control for non-specific staining. These data confirm other studies indicating the expression of denatured forms of collagen can be selectively expressed within tumor blood vessels (7-9).

### Example 2: Expression and Secretion of a Novel RGDKGE (SEQ ID NO: 1)-Containing Collagen Peptide by Malignant Tumor Cells

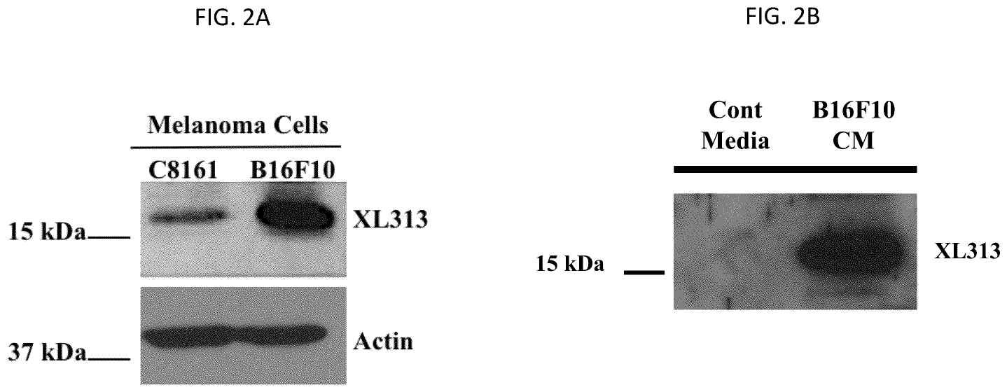

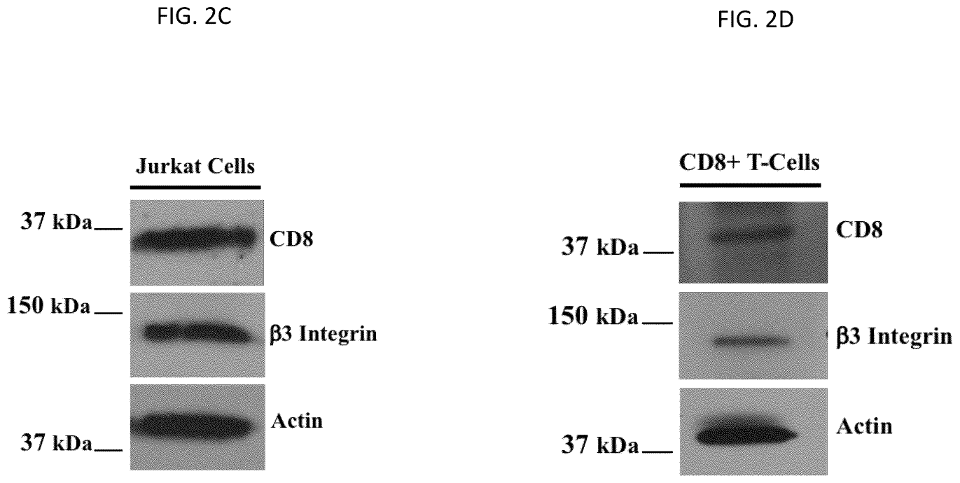

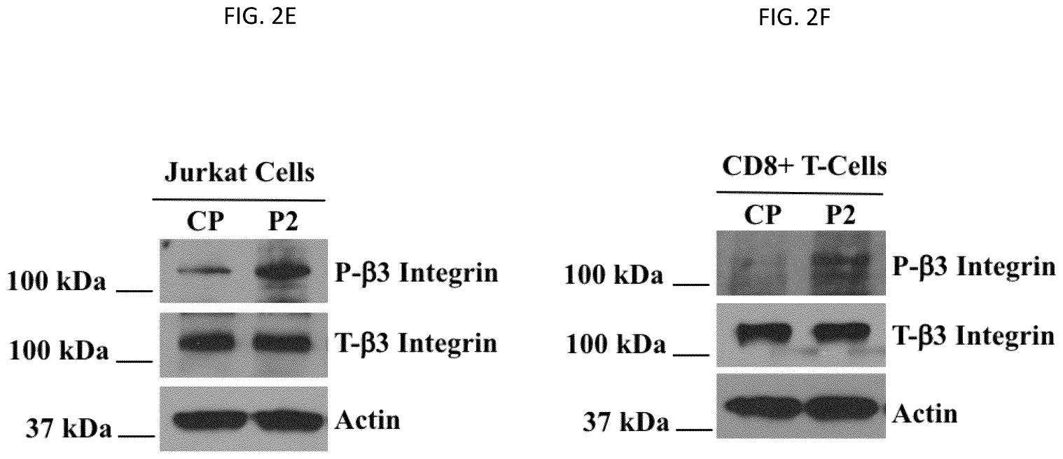

We previously made the surprising observation that a small bioactive collagen peptide containing the core amino acid sequence RGDKGE (SEQ ID NO: 1) could be generated inside a subset of M2-like activated macrophages as well as a variety of other malignant tumor cells, and then actively secreted outside the cells (11-13). Importantly, minimal levels of this soluble collagen peptide was detected in normal cells (13). We confirmed these observations by examining the expression of the low molecular weight 16 kDa RGDKGE (SEQ ID NO: 1)-containing collagen peptide by western blot of whole cell lysates and serum free conditioned medium in melanoma cell lines. As shown in FIG. 2A, the XL313 collagen peptide could be detected in a number of different melanoma cell types as well as serum free conditioned medium from B16F10 melanoma cells (FIG. 2B). Given our previous work indicating that the small RGDKGE (SEQ ID NO: 1)-containing collagen peptide could bind to the cell surface integrin β3 and activate a signaling cascade that regulated angiogenesis and tumor growth in vivo (11-13), we sought to determine whether this collagen peptide might also bind to β3 integrin and regulate the behavior of T-cells. Previous studies have shown that activated T-cells can express β3 integrin (14-16). To confirm these findings, we first examined whether human Jurkat T-cells expressed the β3 integrin receptor that is capable of binding to the soluble collagen peptide. As shown in FIG. 2C, human Jurkat T-cells not only express CD8, a critical marker of CD8+ effector T-cells, but also expressed β3 integrin as indicated by western blot. Using a similar strategy, we isolated primary CD8+ T-cells from melanoma bearing mice and demonstrated that these tumor associated CD8+ T-cells also expressed β3 integrin (FIG. 2D). Given these observations, we next stimulated human Jurkat T-cells or primary CD8+ T-cells with a synthetic version of the soluble RGDKGE (SEQ ID NO: 1)-containing collagen peptide termed P2 (11-13) and demonstrated that this collagen peptide induced activation of β3 integrin as indicated by enhanced β3 integrin phosphorylation as compared to a non-specific control peptide (CP) lacking the RGDKGE (SEQ ID NO: 1) sequence (FIGS. 2E and 2F). Collectively, these studies indicate that the RGDKGE (SEQ ID NO: 1) collagen peptide P2 can bind to T-cells and stimulate downstream signaling in these T-cells.

### Example 3: Treatment of CD8+ T-Cells with the Soluble RGDKGE (SEQ ID NO: 1)-Containing Collagen Peptide (P2) Inhibits Cell Polarization on Denatured Collagen-Type-IV

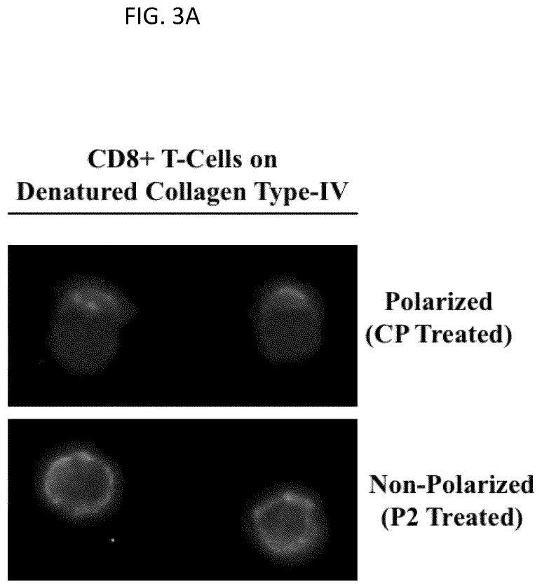

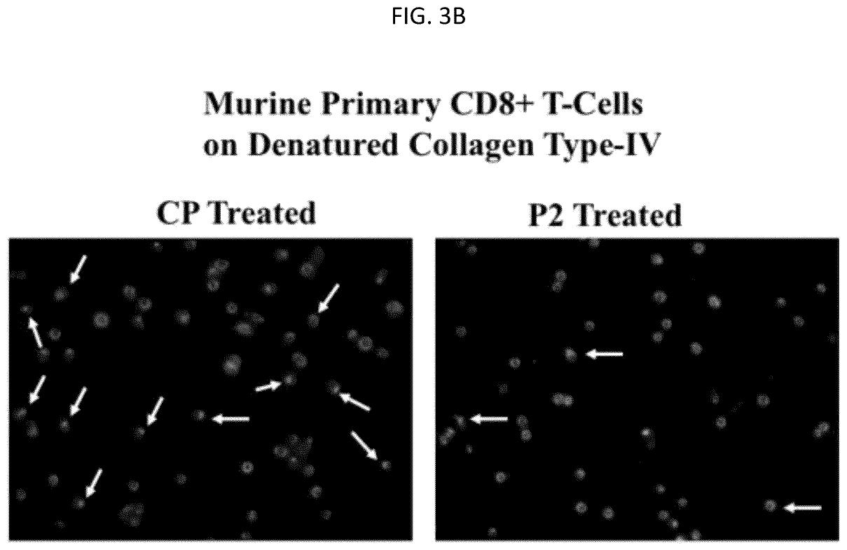

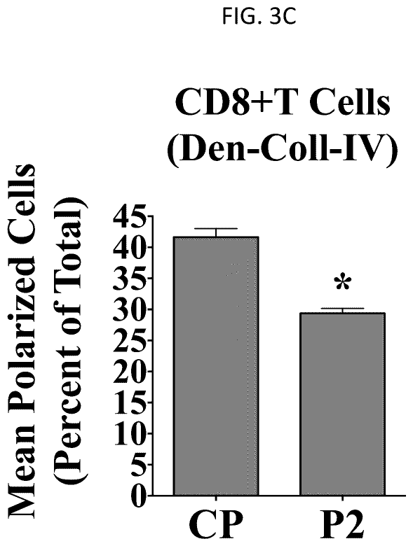

T-cell are thought to utilize a type of cell motility called ameboid movement during trans-endothelial cell migration through the endothelial cell layer of blood vessels. Importantly, this type of cell migration involves the reorganization of the actin cytoskeleton in such a way as to result in the formation of uropods or protrusions on one side of the cell, resulting in a morphological change in the shape of the T-cells allowing them to become polarized to facilitate directed cell movement. Thus, it has been suggested that T-cell polarization is a characteristic of motile T-cells (17,18). Given that denatured collagen type-IV is selectively enriched in the basement membranes of tumor vessels, we examined whether the soluble RGDKGE (SEQ ID NO: 1)-containing collagen peptide P2 might alter T-cell morphology. Briefly, we coated microscope slides with denatured collagen type-IV to mimic the altered basement membrane structures observed with tumor vessels and treated freshly isolated primary murine CD8+ T-cells with the soluble RGDKGE (SEQ ID NO: 1)-containing collagen peptide P2 or non-specific control peptide CP and seeded the cells on the denatured collagen type-IV coated slides for 1 hour. The slides were next washed, fixed and stained with Rhodamine-labeled Phalloidin to visualize F-actin and polarization. An enlarged image (FIG. 3A) is shown as an example of the distinct morphological change in cell shape and differential F-actin localization (red) that is characteristic of polarized CD8+ T-cells. As shown in FIG. 3B, CD8+ T-cells treated with the RGDKGE (SEQ ID NO: 1) collagen peptide P2 exhibited fewer polarized cells as compared to the T-cells incubated with the control peptide CP (white arrows). Importantly, quantification of two independent experiments indicated that treatment of CD8+ T-cells with the RGDKGE (SEQ ID NO: 1) collagen peptide resulted in a significant decrease in the percentage of T-cells exhibiting a migratory and polarized morphological phenotype (FIG. 3C). These exciting studies are consistent with a potential role for the soluble RGDKGE (SEQ ID NO: 1) collagen peptide that can be secreted from immune suppressive macrophages and multiple tumor cell types to alter T-cell behavior on denatured collagen type-IV.

### Example 4: The Soluble RGDKGE (SEQ ID NO: 1)-Containing Collagen Peptide (P2) Selectively Inhibits T-Cell Migration on Denatured Collagen Type-IV, but not on Native Collagen Type-IV

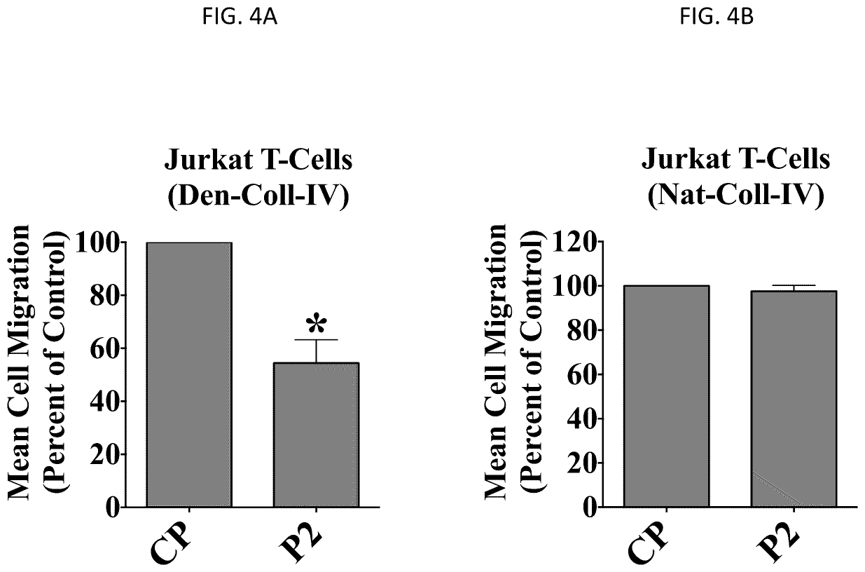

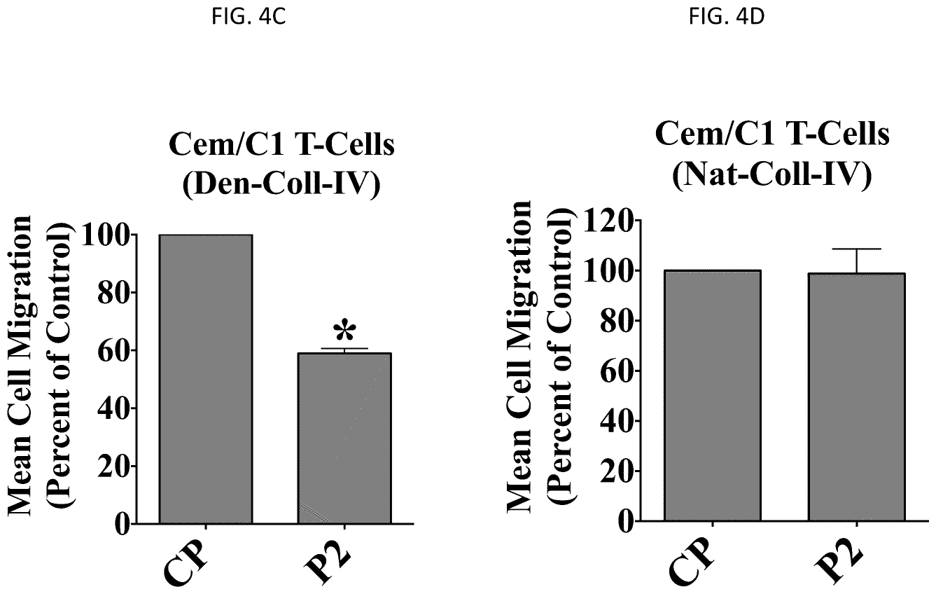

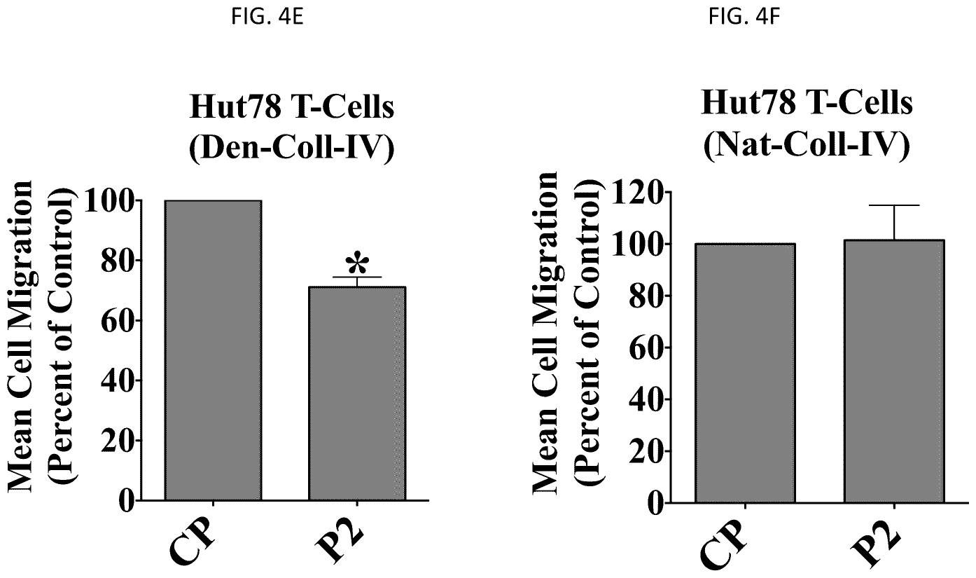

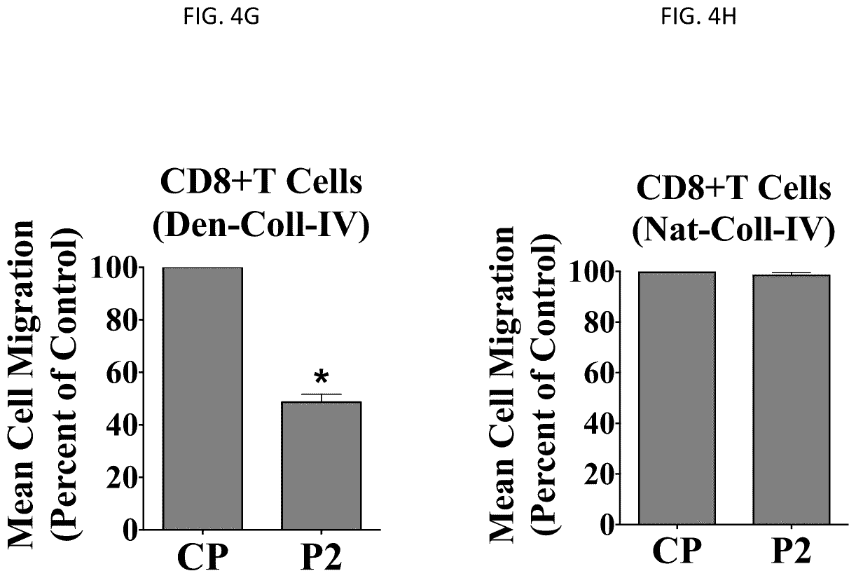

Given our new observations, and the fact that many tumor types as well as immunosuppressive macrophages can secrete the small soluble RGDKGE (SEQ ID NO: 1) collagen peptide, we next sought to determine whether this soluble collagen peptide could impact T-cell migration through denatured collagen type-IV, a critical ECM component shown to be selectively enriched in tumor vessels. Briefly, membranes from transwell migration chambers were coated with either native collagen type-IV or denatured collagen type-IV. Next, various T-cell lines were resuspended in the presence of a non-specific control peptide (CP) or the RGDKGE (SEQ ID NO: 1) collagen peptide P2 and cells (Jurkat and Cem/C1) were allowed to migrate in the presence of the chemoattractant SDF-1 added to the lower chamber. Similar experiments were carried out using the T-cell line Hut78 in the absence of SDF-1. Cell migration was quantified following staining cells with crystal violet (6,8). As shown in FIGS. 4A-4F, neither the soluble RGDKGE (SEQ ID NO: 1) collagen peptide P2, nor the non-specific control peptide had any significant effect on the migration of different T-cell lines (Jurkat, Cem/C1, and Hut78) on native collagen type-IV. In striking contrast, the soluble RGDKGE (SEQ ID NO: 1)-containing collagen peptide P2, but not the control peptide CP, selectively inhibited T-cell migration on denatured collagen type-IV (FIGS. 4A-4F). Finally, we carried out a similar set of experiments using freshly isolated primary CD8+ T-cells from melanoma bearing C57BL/6 mice. As shown in FIGS. 4G-4H, while neither the RGDKGE (SEQ ID NO: 1) collagen peptide P2, nor the non-specific control peptide CP had any significant impact on primary murine CD8+ T-cell migration on native collagen type-IV typically found within normal blood vessels, the RGDKGE (SEQ ID NO: 1) collagen peptide P2, selectively inhibited CD8+ T-cell migration on denatured collagen type-IV as compared to control peptide CP. Taken together, these surprising results indicate that a small soluble RGDKGE (SEQ ID NO: 1)-containing collagen peptide, shown previously to be actively secreted from a number of different malignant tumor cells, can selectively inhibit the migration of T-cell on denatured collagen type-IV.

### Example 5: The Anti-RGDKGE (SEQ ID NO: 1)-Containing Collagen Peptide Antibody Mab XL313 Selectively Enhances T-Cell Migration on Denatured Collagen Type-IV but not on Native Collagen Type-IV

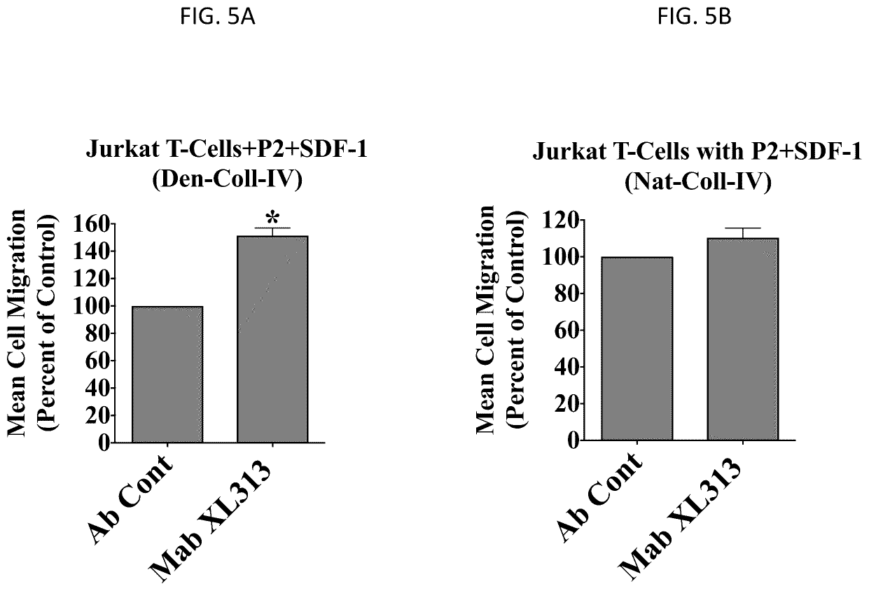

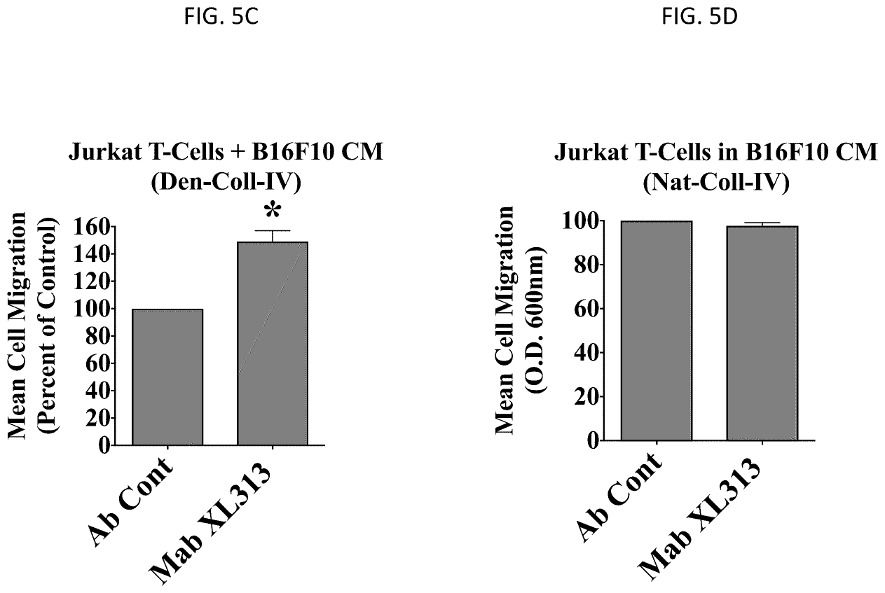

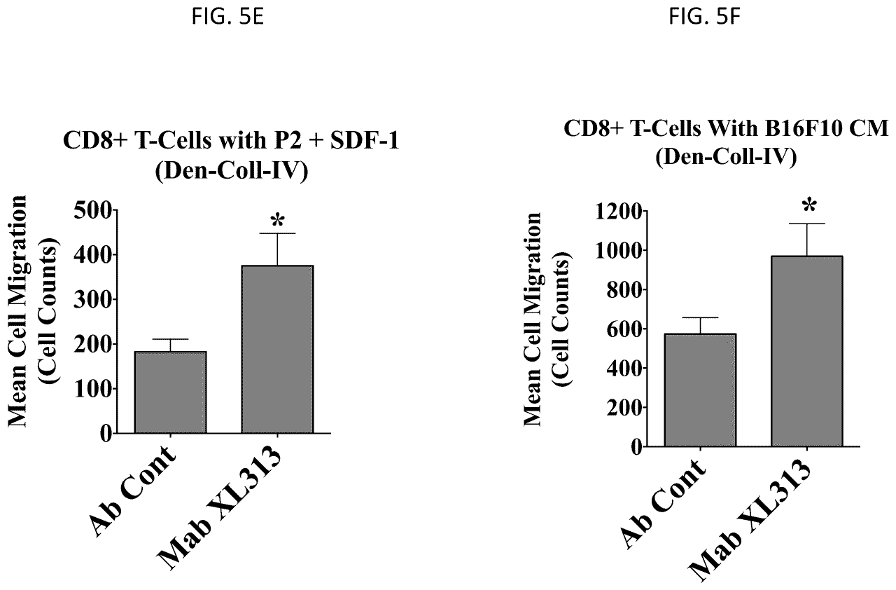

Given our surprising observations that the secreted RGDKGE (SEQ ID NO: 1) collagen peptide can selectively inhibit T-cell migration on denatured collagen type-IV, but not on native collagen type-IV, we sought to determine whether our anti-XL313 antibody specifically directed to the soluble RGDKGE (SEQ ID NO: 1) collagen peptide might reverse the T-cell inhibitory activity selectively on denatured collagen type-IV. To test this possibility, membranes from transwell migration chambers were coated with native or denatured collagen type-IV. Next, T-cell lines were incubated with collagen peptide P2 and then, T-cells were resuspended in the presence of a non-specific control antibody (Ab cont) or Mab XL313 and cells were allowed to migrate in the presence of the chemoattractant SDF-1 in the lower chamber. As shown in FIG. 5A, Mab XL313 significantly enhanced Jurkat T-cell migration on denatured collagen type-IV as compared to the non-specific control antibody. Importantly, Mab XL313 failed to significantly impact T-cell migration on native collagen type-IV (FIG. 5B). In further studies, we carried out additional experiments in which we used B16F10 conditioned medium as the source of the RGDKGE (SEQ ID NO: 1)-containing collagen peptide, since our previous studies confirmed that this secreted collagen peptide was generated by B16F10 melanoma cells (13). As expected, while Mab XL313 had no effect of Jurkat T-cell migration on native collagen type-IV as compared to control antibody (FIG. 5D), Mab XL313 significantly enhanced Jurkat T-cell migration on denatured collagen type-IV (FIG. 5C). Finally, using a similar strategy, we examined the effects of Mab XL313 on primary CD8+ T-cells isolated from mice harboring a malignant melanomas. As shown in FIG. 5E, again Mab XL313 significantly enhanced CD8+ T-cell migration on denatured collagen type-IV as compared to control antibody. Importantly, similar results were also observed when using B16F10 melanoma cell conditioned medium as the source of the collagen peptide (FIG. 5F). These surprising new studies provide evidence for the first time, that a unique antibody specifically directed to the secreted RGDKGE (SEQ ID NO: 1)-containing collagen peptide can result in enhanced T-cell migration through denatured collagen type-IV, but not through native collagen type-IV.

### Example 6: Selective Depletion of CD8+ T-Cells from Mice Enhance B16F10 Melanoma Growth In Vivo

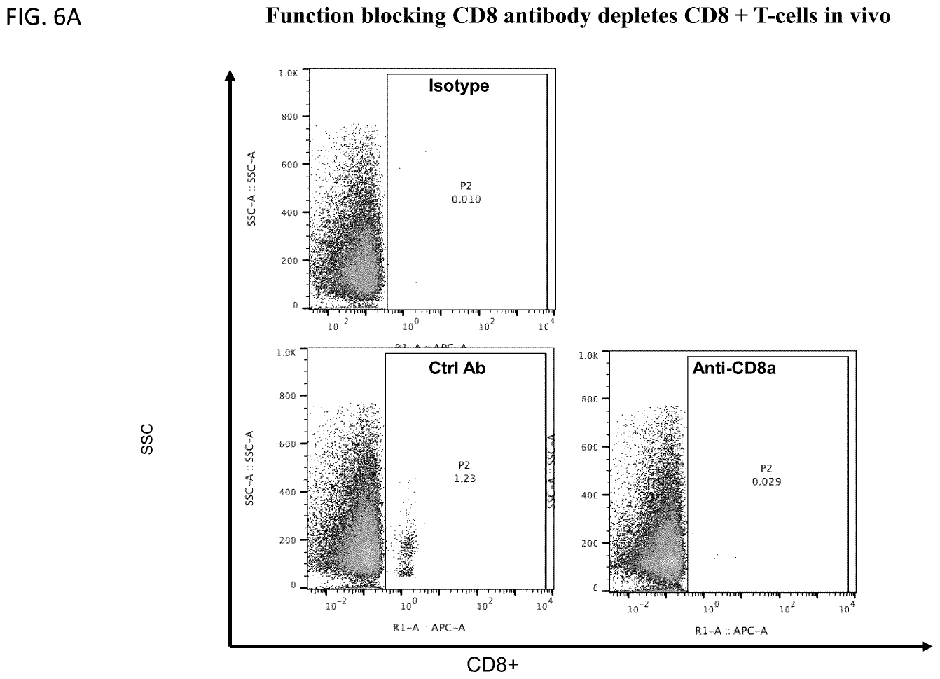

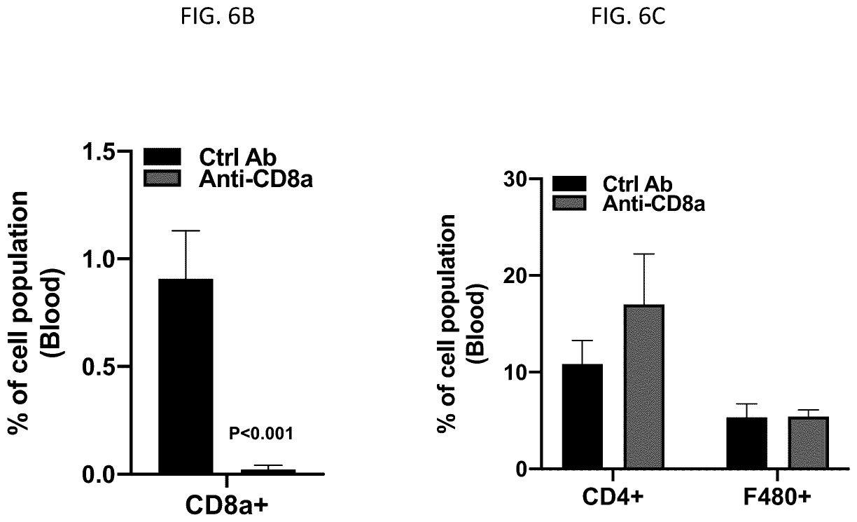

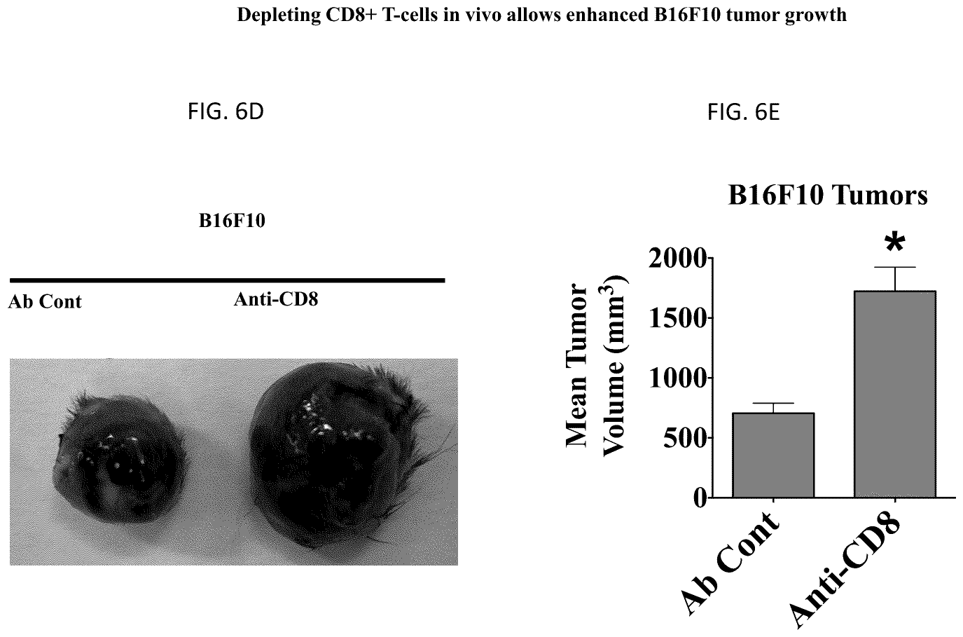

Previous studies have suggested that CD8+ T-cells play a functional role in controlling the growth of multiple types of malignant tumors. To establish a model in which to examine the relevance of CD8+ T-cell in regulating the ability of Mab XL313 to inhibit tumor growth, we depleted CD8+ T-cells from C57BL/6 mice. As shown in FIGS. 6A-6C, treatment of C57BL/6 mice with function blocking anti-CD8 efficiently depleted CD8+ T-cells, while having no significant depleting impact on other immune cells such as CD4+ T-cells or F/480+ macrophages. Next, we confirmed the tumor growth promoting activity of selectively reducing CD8+ T-cells from mice on B16F10 melanomas growing in vivo. As expected, the growth of B16F10 melanomas were significantly higher in mice lacking CD8+ T-cell (FIG. 6A-6E), confirming the important role of CD8+ T-cells in controlling melanoma tumor growth in vivo. These important data help provide a functional in vivo model to test the impact of CD8+ T-cells on the anti-tumor activity of Mab XL313.

### Example 7: The Anti-Tumor Activity of Mab XL313 Depends on the Levels of Tumor Infiltrating CD8+ T-Cells In Vivo

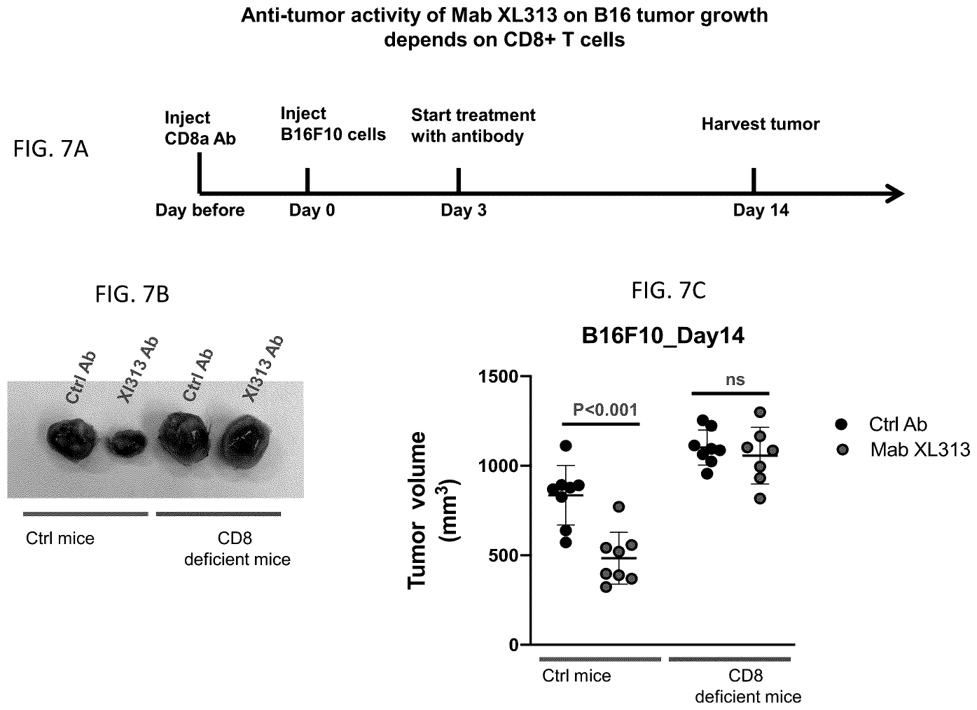

Our new data indicate that the control of B16F10 melanoma tumor growth in mice depends in part on the levels of CD8+ T-cell infiltration of tumors. Thus, we next sought to examine whether the ability of Mab XL313 to control tumor growth also depends at least in part on the levels of CD8+ T-cells. In the regard, we first treated C57BL/6 mice with anti-CD8+ antibody to deplete CD8+ T-cells or treated them with non-specific control antibody. Next, mice were treated with Mab XL313 or control antibody, and tumor growth was monitored over a 14 day time course, for 8 mice per experimental condition. As shown in FIGS. 7A-7C, while the growth of B16F10 melanoma tumors in animals subjected to a control immune depletion protocol were significantly inhibited by Mab XL313, Mab XL313 failed to inhibit tumor growth in mice in which CD8+ T-cells were depleted. These important studies indicate that the ability of Mab XL313 to inhibit B16F10 tumor growth depends on the levels of CD8+ T-cells.

### Example 8: Mab XL313 Significantly Enhances Infiltration of CD8+ T-Cells in Tumors

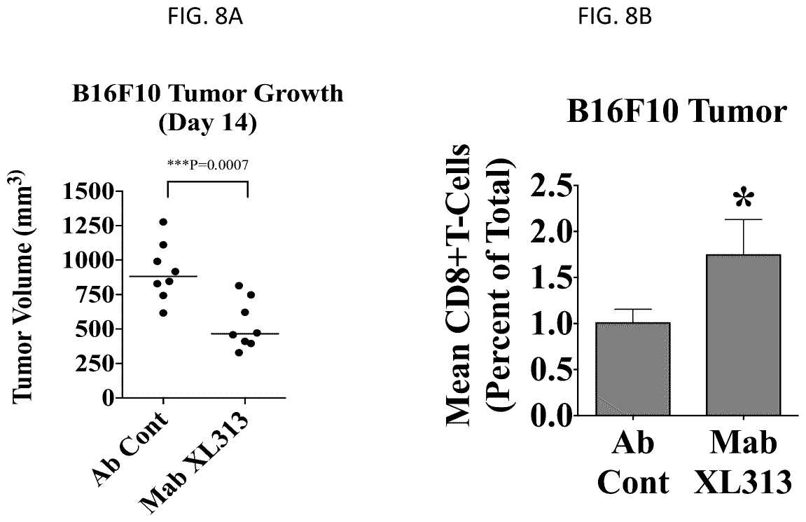

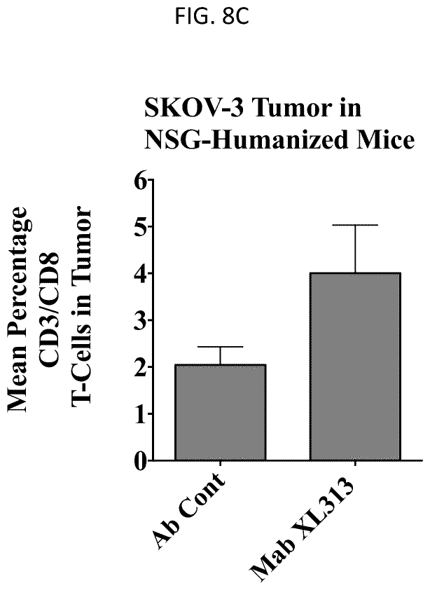

Given the critical role for high levels of T-cell migration through tumor vessels in sufficient numbers for effective ACT, and our exciting new findings that Mab XL313 can selectively enhance T-cell migration through denatured collagen type-IV, which is specifically enriched in tumor vessels, we examined the ability of Mab XL313 to enhance T-cell infiltration of tumors in vivo. As shown in FIG. 8A, consistent with our previous studies (13), treatment of mice with Mab XL313 significantly inhibited B16F10 melanoma tumor growth. Surprisingly, tumors from mice treated with Mab XL313 exhibited significantly enhanced levels of CD8+ T-cells infiltrating the tumors as compared to control antibody treatment (FIG. 8B). Similar results were also observed using a humanized mouse model in which human SKOV-3 ovarian carcinoma tumors were growing. As shown in FIG. 8C, treatment of mice with Mab XL313 resulted in enhanced infiltration of CD8+ T-cells into these ovarian tumors. Collectively, these surprising observations, provide evidence for the first time that a unique antibody (Mab XL313) has the ability to selectively enhance the infiltration of tumor killing CD8+ T-cells into tumors growing in vivo, and provides the basis for a new strategy to selectively enhance the efficacy of ACT based on its potent and selective activity in vivo.

### Example 9: T-Cell Lines as Well as Primary T-Cells Isolated from B16F10 Tumor Bearing Mice Exhibit Reduced Migration on Denatured Collagen-IV Compared to Native Collagen-IV

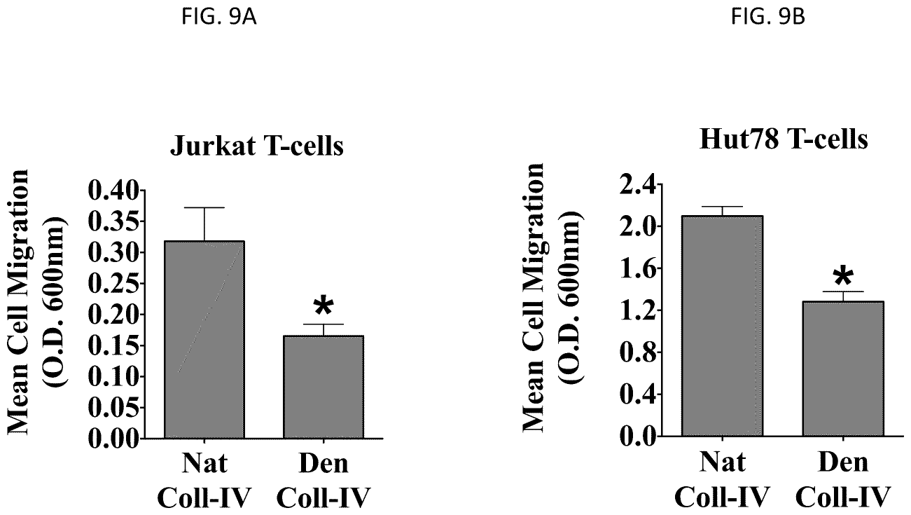

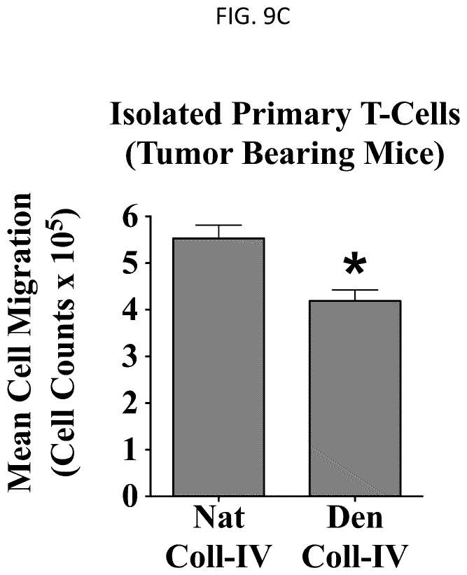

While much is known concerning the molecular mechanisms that regulate T-cell transendothelial migration, much less is known concerning how T-cells migrate through the restrictive vascular subendothelial basement membrane of tumor vessels that are rich in denatured forms of collagen type-IV. Once T-cells have migrated through the common endothelial cell layer of blood vessels, they must traverse the highly restrictive underlying vascular basement membrane. Importantly, tumor vessel basement membranes have been shown to be quite different than basement membranes associated with normal vessels as they are often enriched with structurally altered denatured collagen. Therefore, we compared the ability of T-cells to migrate on either native collagen type-IV or denatured collagen type-IV. To facilitate these studies, membranes from transwell migration chambers were coated with native or denatured collagen type-IV. Next, T-cell lines were allowed to migrate in the presence of the chemoattractant SDF-1 in the lower chamber. As shown in FIG. 9A and FIG. 9B, migration of human T-cell lines Jurkat and Hut78 were significantly reduced on denatured collagen type-IV as compared to native collagen type-IV. Importantly, using similar assays, isolated primary T-cells from B16F10 tumor bearing mice also showed a similar reduction in migratory ability on denatured collagen type-IV (FIG. 9C).These data are consistent with the notion that T-cell interaction with the denatured collagen type-IV present within the vascular basement membranes of tumor blood vessels may restrict T-cell migration.

### Example 10: The Mab Anti-HU177 Antibody Enhances T-Cell Migration on Denatured Collagen Type-IV

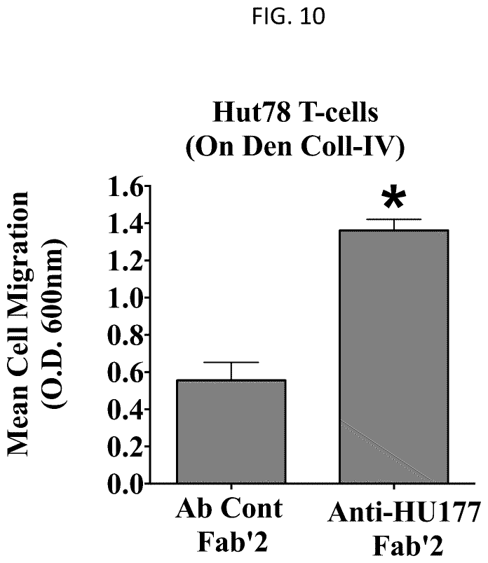

Given our surprising observations that denatured forms of collagen type-IV can selectively reduce T-cell migration, we sought to determine whether our anti-HU177 antibody that is specifically directed to denatured forms of collagen, might reverse the inhibitory activity. To test this possibility, membranes from transwell migration chambers were coated with denatured collagen type-IV. Next, Hut78 T-cells we were resuspended in the presence of fab fragments of non-specific control antibody (Ab cont.) or Mab HU177 and cells were allowed to migrate. As shown in FIG. 10, Mab HU177 significantly enhanced Hut78 T-cell migration on denatured collagen type-IV.

### Example 11: The Mab Anti-HU177 Antibody Enhances the Levels of CD8+ T-Cells in Malignant Ascites Fluid In Vivo

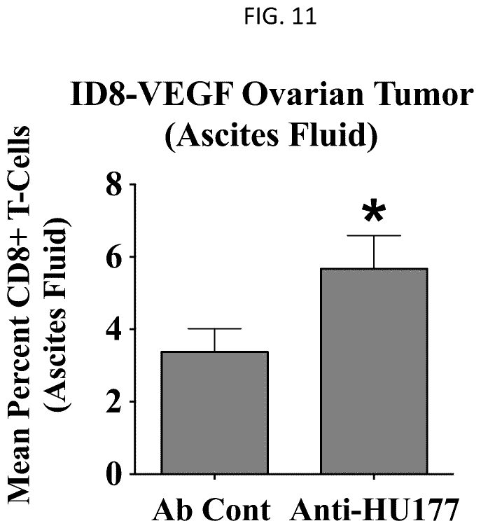

Given the surprising results that the Mab HU177 enhanced T-cell migration on denatured collagen type-IV, we next evaluated if administration of HU-177 antibody could increase the levels of CD8+ T-cells found in malignant ascites fluid. To this end, C57BL/6 mice were first injected intraperitoneally with ID8-VEGF ovarian tumor cells, following which the tumor cells were allowed to grow within the peritoneal cavity of mice for 4 weeks to allow the formation of malignant ascites fluid and solid tumors. Beginning at 4 weeks, mice were injected subcutaneously twice a week with 100 μg/mouse of either non-specific control antibody (Ab Cont) or anti-HU177 antibody (Anti-HU177). At 12 weeks, mice were sacrificed and malignant ascites fluid was collected. The relative levels of CD8+ T-cells within the malignant ascites fluid was quantified by flow cytometry, for 4 mice per experimental condition. As shown in FIG. 11, administration of the Anti-HU-177 antibody resulted in a significant increase in the mean percentage of CD8+ T-cells measured in the ascites fluid, as compared to administration of the control antibody.

### Example 12: CD4+ Treg Cells Isolated from B16F10 or ID8-VEGF Tumor Bearing Mice Exhibit Enhanced Migration on Denatured Collagen-IV as Compared to Native Collagen-IV

It is known that the balance between cytotoxic CD8+ T-cells and immunosuppressive CD4+ Treg as well as myeloid derived suppressor cells (MDSC) cells plays a critical role in the efficacy of immunotherapy. In fact, increasing the relative levels of CD8+ T-cells while reducing the levels of immunosuppressive cells (CD4+ Tregs and MDSC) would represent an approach to enhance the therapeutic activity of immunotherapy including adoptive cell therapy. However, developing strategies to accomplish this is highly challenging. To this end, while immunosuppressive CD4+ Tregs as well as MDSC cells play a role in reducing the levels and cytotoxic activity of CD8+ T-cell infiltration into tumors, it is not known whether denatured collagen-IV, enriched in the basement membranes of tumor associated vessels alters immunosuppressive cell migration.

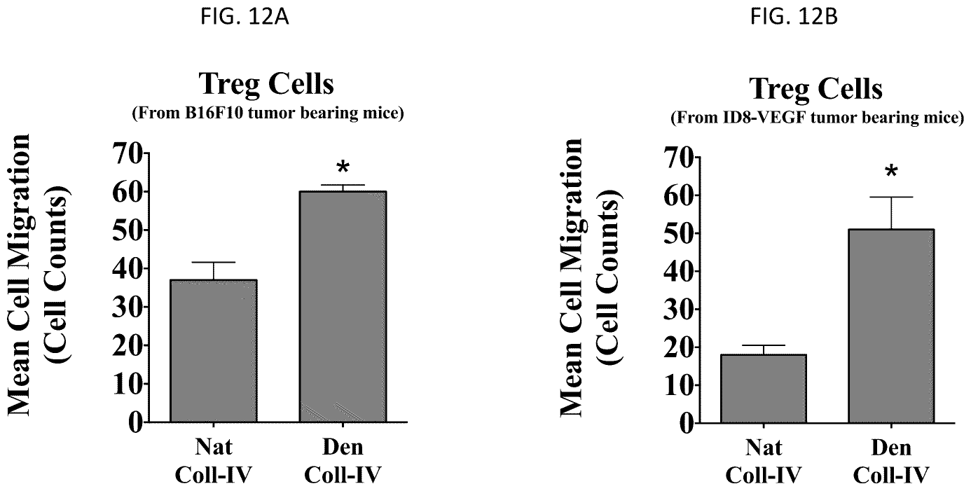

Therefore, we next compared the ability of regulatory T-cells (Treg) to migrate on either native collagen type-IV or denatured collagen type-IV. For this experiment, CD4+ Treg cells were isolated from either B16F10 or ID8-VEGF tumor bearing mice. Briefly, mice (C57BL/6) were either injected subcutaneously with B16F10 cells, or intraperitoneally with ID8-VEGF cells. The tumors were allowed to grow for 14-days. Spleens from the tumor bearing mice were dissected and primary murine CD4+ Treg cells were isolated and analyzed for migration on either native collagen type-IV or denatured collagen type-IV. As shown in FIGS. 12A-12B, the migration of CD4+ Treg cells isolated from B16F10 or ID8-VEGF tumors was significantly increased on denatured collagen type-IV as compared to native collagen type-IV. Together, these studies provide surprising and unexpected new evidence that CD4+ Treg cell migration is enhanced on denatured collagen-IV as compared to native collagen-IV. Furthermore, given that denatured collagen-IV, which binds to integrin 100, has been shown to be highly enriched within tumor associated blood vessels, the enhanced migration of CD4+ Treg cells may lead to enhanced infiltration of tumors, immune suppression and reduced efficacy of adoptive cell therapy.

### Example 13: Stimulation of CD4+ Treg Cells with the Soluble RGDKGE (SEQ ID NO: 1) Collagen Peptide (P2) Enhances Migration on Denatured Collagen-IV as Compared to Stimulation with Control Peptide (CP)

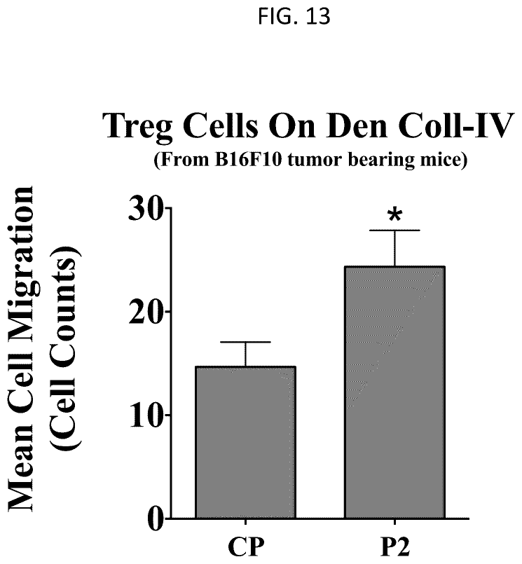

Given our surprising finding that CD4+ Treg cell migration is enhanced on denatured collagen-IV as compared to native collagen-IV, we next tested whether CD4+ Treg cell migration through denatured collagen-IV is altered by endogenously generated soluble RGDKGE (SEQ ID NO: 1) collagen peptide P2. For these experiments, mice (C57BL/6) were injected subcutaneously with B16F10 cells. B16F10 tumors were allowed to grow for 14-days to allow formation of solid malignant melanomas. Spleens from tumor bearing mice were dissected and primary murine CD4+ Treg cells were isolated and analyzed for the effects of the soluble collagen peptide (P2) or control peptide (CP) on cell migration on denatured collagen type-IV. As shown in FIG. 13, the presence of the soluble collagen P2 peptide caused a significant increase in the mean cell migration cell count, as compared to migration in the presence of control peptide (CP). Together, our T-cell migration experiments show that while the RGDKGE (SEQ ID NO: 1) collagen fragment can selectively reduce the migration of CD8+ T-cells on denatured collagen-IV, this RGDKGE (SEQ ID NO: 1) collagen peptide has the opposite effect and enhanced Treg cell migration on denatured collagen-IV. Collectively, these new studies provide surprising and unexpected findings that support the concept that the RGDKGE (SEQ ID NO: 1) collagen peptide has a dual effect on regulating T-cells migration by inhibiting CD8+ T-cell migration while enhancing Treg cell migration.

### Example 14: Anti-HU177 Antibody Reduces the Levels of CD4+ Treg Cells in Ascites Fluid from Mice with ID8-VEGF Ovarian Tumors

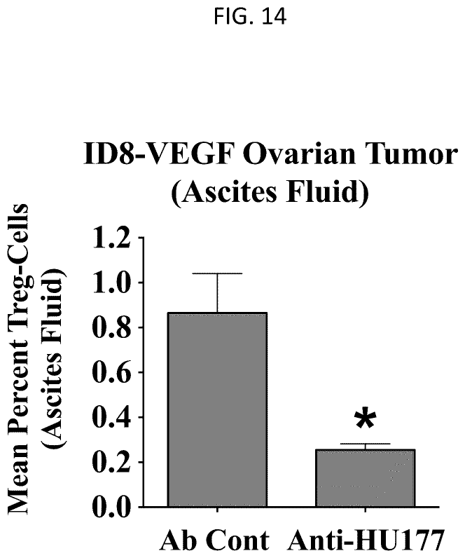

Given that selective targeting of denatured collagen with Mab HU177 can inhibit denatured collagen binding to integrin α10, it is likely that blocking denatured collagen-integrin α10 interactions, will also differentially alter the balance of cytotoxic CD8+ T-cell and immunosuppressive cells such as Tregs and MDSC. To this end, we quantified the relative levels of CD4+ Tregs in ovarian tumor ascites, upon treatment with either anti-HU177 antibody (Anti-HU177), or non-specific control antibody (Ab Cont). Briefly, mice (C57BL/6) were injected intraperitoneally with ID8-VEGF ovarian tumor cells. ID8-VEGF tumor cells were allowed to grow within the peritoneal cavity of mice for 4-weeks to allow formation of malignant ascites fluid and solid tumors. Beginning at 4 weeks, mice were injected subcutaneously twice a week with 100 μg per mouse non-specific control antibody (Ab Cont) or anti-HU177 antibody (Anti-HU177). At 12 weeks, mice were sacrificed and malignant ascites fluid was collected. The relative levels of CD4+ Treg-cells within the malignant ascites fluid was quantified by flow cytometry. The quantification of CD4+ Treg-cells showed a significant decrease in mean % Treg cells upon treatment with anti-HU177 antibody as compared to control antibody (FIG. 14). Therefore, these surprising results show that treatment of mice with anti-HU177 Mab resulted in reduced levels of CD4+ Tregs as compared to control.

### Example 15: Modulation of Levels of Myeloid-Derived Suppressor Cells (MDSC) and CD8+ T-Cells in B16F10 Melanoma Tumors Growing in Integrin α10 Knockdown Mice

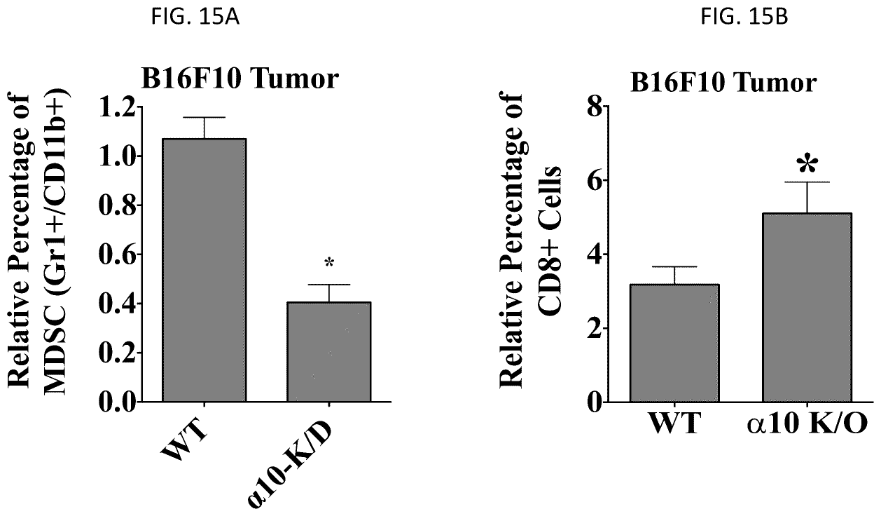

In order to assess whether Mab HU177, which blocks the ability of denatured collagen to bind to integrin α10, may also affects MDSCs, we next quantified the levels of cytotoxic CD8+ T-cells and immunosuppressive MDSC in mice tumors growing in integrin α10 knockdown mice. For these experiments, wild type integrin α10 expressing mice (WT), and integrin α10 knockdown mice (α10-K/D) were injected with equal numbers of B16F10 melanoma cells and allowed to form tumor for 14 days. Tumors were then dissected and single cell suspensions were prepared and analyzed for the relative levels of MDSCs and CD8+ T-cells from each condition. As shown in FIGS. 15A-15B, the relative percentage of MDSCs were significantly reduced in B16F10 tumors in α10 knockdown mice compared to wild-type, whereas the relative levels of CD8+ cells was significantly increased. Thus, these surprising new findings indicate that tumors growing in mice with reduced levels of integrin α10, which binds denatured collagen, have elevated levels of CD8+ T-cells and reduced levels of immunosuppressive MDSC.

### Example 16: Selective Targeting of the RGDKGE (SEQ ID NO: 1) Collagen Peptide P2 Reduces the Levels of Activated/Phosphorylated P38MAPK

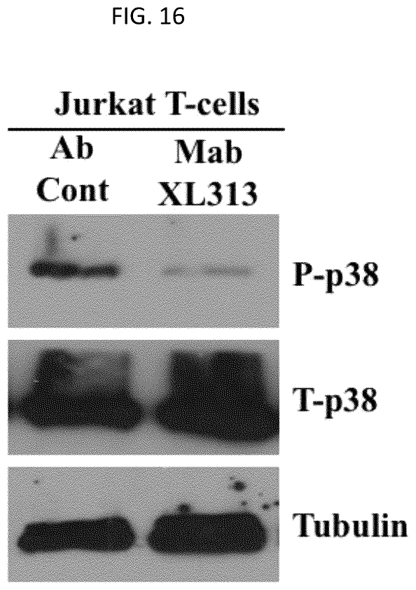

Our previous studies (19) suggested that the soluble RGDKGE (SEQ ID NO: 1)-collagen peptide P2 can enhance activation of an important MAP kinase, i.e., the phosphorylation of P38MAPK in endothelial cells. Recent evidence indicates that activated P38MAPK may play a critical role limiting the efficacy of adoptive cell therapy, while inhibiting activation/phosphorylation of P38MAPK may enhance efficacy of adoptive cell therapy (20). Thus, we examined the effects that targeting the soluble RGDKGE (SEQ ID NO: 1)-collagen peptide P2 had on P38MAPK phosphorylation in human CD8+ Jurkat T-cell. First, we preincubated the RGDKGE (SEQ ID NO: 1) collagen peptide P2 with non-specific control antibody (Ab Control) or anti-RGDKGE (SEQ ID NO: 1) Mab XL313 to inhibit P2 for 30 minutes, following which we stimulated Jurkat T-cells under each of these conditions for 15 minutes. Whole cell lysates were prepared and Western blots were used to analyze levels of phosphorylated P38MAPK. As shown in FIG. 16, selective targeting of the RGDKGE (SEQ ID NO: 1) collagen peptide P2 inhibited the levels of activated /phosphorylated P38MAPK as compared to control antibody in these T-cells. Therefore, these data provide surprising and unexpected new evidence that selective targeting of the RGDKGE (SEQ ID NO: 1) collagen peptide can inhibit a key molecule thought to limit adoptive cell therapy.

### REFERENCES

1). Morotti, M., Albukhari, A., Alsaadi, A., Aribani, M., Brenton, J. D., Curbishley, S. M., Dong, T., Dustin, M. L., Ju, Z., McGranahan, N., Miller, M. L., Santana-Gonzalez, L., Seymour, L. W., Shi, T., Loo, P. V., Yau, C., White, H., Wietek, N., Church, D. N., Wedge, D. C., and Ahmed, A. A. Promises and challenges of adoptive T-cell therapies for solid tumors. Br. J. Cancer 124: 1759-1776. 2021.
2). Nguyen, D. T., Ogando-Rivas, E., Liu, R., Wang, T., Rubin, J., Jin, L., Tao, H., Sawyer, W. W., Mendez-Gomez, H. R., Cascio, M., Mitchell, D. A., Huang, J., Sawyer, W. G., Sayour, E. J., and Castillo, P. CAR T cell locomotion in solid tumor microenvironment. Cells. 11: 1974. doi.org/10.3390/cells11121974. 2022.
3). Michaelides, S., Obeck, H., Kechur, D., Endres, S., and Kobold, S. Migratory engineering of T cells for cancer therapy. Vacines. 10,1845. Doi.org/10.3390/vaccines10111845. 2022.
4). Kalluri, R. Basement membranes: structure, assembly and role in tumor angiogenesis. Nat. Rev. Cancer. Doi:10.1038/nrc1094. 2003.
5). Xu, J., Rodriguez, D., Petitclerc, E., Kim, J. j., Hangai, M., Moon, Y., Davis, G. E., and Brooks, P. C. Proteolytic exposure of a cryptic site within collagen type-IV is required for angiogenesis and tumor growth in vivo. J. Cell Biol. 154: 1069-1079. 2001.
6). Cretu, A., Roth, J. M., Vaunt, M., Akalu, A., Policarpio, D., Formenti, S., Gagne, P., Liebes, L., and Brooks, P. C. Disruption of endothelial cell interactions with the novel HU177 cryptic collagen epitope inhibits angiogenesis. Clin. Cancer Res. 13: 3068-3078. 2007.
7). Pernasetti, F., Nickel, J., Clark, D., Baeuerle, P. A., Van Epps, D., and Freimark, B. Novel anti-denatured collagen humanized antibody D93 inhibits angiogenesis and tumor growth: an extracellular matrix-based therapeutic approach. Int. J. Oncol. 29: 1371-1379. 2006.
8). Caron, J. M., Ames, J. J., Contois, L., Liebes, L., Friesel, R. E., Vary, C. P. H., Oxburgh, L., and Brooks, P. C. Inhibition of ovarian tumor growth by targeting the HU177 cryptic collagen epitope. AM. J. Pathol: 2016; 186: 1649-1661.
9). Pernasetti, F., Nickel, J., Clark, D., Baeuerle, P. A., Van Epps, D., and Freimark, B. Novel anti-denatured collagen humanized antibody D93 inhibits angiogenesis and tumor growth: an extracellular matrix-based therapeutic approach. Int. J. Oncol. 29: 1371-1379. 2006.
10). Castermans, K., and Griffioen, A. W. Tumor blood vessels, a difficult hurdle for infiltrating leukocytes. Biochim. Biophys. Acta. 1776: 160-174. 2007.
11). Ames, J. J., Contois, L., Caron, J. M., Tweedie, E., Yang, X., Friesel, R., Vary, C., and Brooks, P. C. Identification of an endogenously generated cryptic collagen epitope (XL313) that may selectively regulate angiogenesis by an integrin Yes-associated protein (TAP) mechano-transduction pathway. J. Biol. Chem. 291: 2731-2750. 2016.
12). Han, X., Caron, J. M., Lary, C. W., Sathyanarayana, P., Vary, C., and Brooks, P. C. An RGDKGE-containing cryptic collagen fragment regulates phosphorylation of large tumor suppressor kinase-1 and controls ovarian tumor growth by a yes-associated protein-dependent mechanism. Am. J. Pathol. DOI: 10.1016/j.apath.2020.11.009. 2021.
13). Caron, J. M., Han, X., Lary, C. W., Sathyanarayana, P., Remick, S. C., Ernstoff, M. S., Herlyn, M., and Brooks, P. C. Targeting the secreted RGDKGE collagen fragment reduces PD-L1 y a proteasome-dependent mechanism and inhibits tumor growth. Oncol. Rep. 49: doi: 10.3892/or.2023.848. 2023.
14. Halvorson, M. J., Coligan, J. E. and Sturmhofel, K. The vitronectin receptor (alpha-V beta-3) as an example for the role of integrins in T-lymphocyte stimulation. Immunol Res. 15, 16-29 (1996).
15). Gerber, D. J., Pereira, P., Huang, S. Y., Pelletier, C. and Tonegawa, S. Expression of alpha-V and beta-3 integrin chains on murine lymphocytes. Proc. Natl. Acad. Sci. USA. 93, 14698-14703 (1996).
16). Larochelle, C., Uphaus, T., Broux, B., Gowing, E., Paterka, M., Mihel, L., Dudvarski Stankovic, N., Bicker, F., Lemaitre, F., Prat, A., Schmidt, M. H. H. and Zipp, F. EGFL7 reduces CNS inflammation in mouse. Nat. Commun. 9, 819 (2018).
17). Serrador, J. M. Nieto, M., and Sanchez-Madrid, F. Cytoskeletal rearrangement during migration and activation of T lymphocytes. Trend Cell Biol. 9: 228-233. 1999.
18). Niggli, V. Insight into the mechanism for dictating polarity in migrating T-cells. Int. Rev. Cell Mol. Biol. 312: 201-270. 2013.
19) Ames, J. J., Contois, L., Caron, J. M., Tweedie, E., Yang, X., Friesel, R., Vary, C., and Brooks, P. C. Identification of an endogenously generated cryptic collagen epitope (XL313) that may selective regulates angiogenesis by an integrin-Yes-associated protein (YAP) mechano-transduction pathway. J. Biol. Chem. 2016; 1406:255-269.
20) Devikala Gurusamy, Amanda N. Henning, Tori N. Yamamoto, Zhiya Yu, Nikolaos Zacharakis, Sri Krishna, Rigel J. Kishton, Suman K. Vodnala, Arash Eidizadeh, Li Jia, Christine M. Kariya, Mary A. Black, Robert Eil, Douglas C. Palmer, Jenny H. Pan, Madhusudhanan Sukumar, Shashank J. Patel, Nicholas P. Restifo Multi-phenotype CRISPR-Cas9 Screen Identifies P38 Kinase as a Target for Adoptive Immunotherapies. Cancer Cell. 2020 Jun. 8; 37(6): 818-833.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

We claim:

1. A method of treating cancer in a subject, the method comprising administering to the subject
a) an adoptive cell therapeutic composition; and
b) an antagonist of collagen or a functional fragment thereof,
thereby treating cancer in the subject.

2. A method of increasing the efficacy of adoptive cell therapy or T-cell therapy in a subject having cancer, the method comprising administering to the subject
a) an adoptive cell therapeutic composition; and
b) an antagonist of collagen or a functional fragment thereof,
thereby increasing the efficacy of the adoptive cell therapy or T-cell therapy compared to a control.

3. A method of modulating T-cell infiltration into a tumor in a subject, the method comprising administering to the subject
a) an adoptive cell therapeutic composition; and
b) an antagonist of collagen or a functional fragment thereof,
thereby modulating T-cell infiltration into the tumor in the subject compared to a control.

4. A method of modulating Treg and/or myeloid-derived suppressor cell (MDSC) levels in a tumor in a subject, the method comprising administering to the subject
a) an adoptive cell therapeutic composition; and
b) an antagonist of collagen or a functional fragment thereof,
thereby modulating Treg and/or myeloid-derived suppressor cells (MDSCs) levels in the tumor in the subject compared to a control.

5. The method of claim 3, wherein the T-cell infiltration into the tumor of the subject is increased compared to the control.

6. The method of claim 4, wherein the Treg and/or MDSC levels are decreased in the tumor in the subject compared to the control.

7. The method of any one of claims 2-4, wherein the control is prior to administration of the adoptive cell therapeutic composition, and the antagonist of collagen or the functional fragment thereof.

8. The method of any one of claims 1-7, wherein the adoptive cell therapeutic composition comprises a cell type selected from a group consisting of a tumor infiltrating lymphocytes (TIL), T-cell receptor modified lymphocytes and chimeric antigen receptor modified lymphocytes.

9. The method of claim 8, wherein the adoptive cell therapeutic composition comprises tumor infiltrating lymphocytes (TIL).

10. The method of any one of claims 1-9, wherein the adoptive cell therapeutic composition comprises a cell type selected from a group consisting of T-cells, CD8+ cells, CD4+ cells, NK-cells, delta-gamma T-cells, regulatory T-cells, and peripheral blood mononuclear cells.

11. The method of claim 10, wherein the adoptive cell therapeutic composition comprises T-cells.

12. The method of any one of the preceding claims, wherein the collagen comprises collagen type-I, collagen type II, collagen type III, or collagen type-IV.

13. The method of claim 12, wherein the antagonist of collagen and functional fragment thereof comprises an antagonist of collagen type-I or a functional fragment thereof, or an antagonist of collagen type-IV or a functional fragment thereof.

14. The method of claim 13, wherein the antagonist of collagen type-I or the functional fragment thereof comprises an antagonist of the XL313 cryptic collagen epitope, or an antagonist of the HU177 cryptic collagen epitope.

15. The method of claim 14, wherein the antagonist of collagen type-I or the functional fragment thereof comprises an antibody or an antigen-binding fragment thereof that binds a cryptic RGDKGE (SEQ ID NO: 1) containing collagen epitope.

16. The method of claim 14, wherein the antagonist of collagen type-I or the functional fragment thereof comprises an antibody or an antigen-binding fragment thereof that binds a cryptic CPGFPGFC (SEQ ID NO: 16) containing collagen epitope.

17. The method of claim 15 or 16, wherein the antibody or the antigen-binding fragment thereof comprises a monoclonal antibody or an antigen-binding fragment thereof.

18. The method of claim 17, wherein said monoclonal antibody or antigen-binding fragment thereof comprises an XL313 monoclonal antibody or an antigen-binding fragment thereof.

19. The method of claim 17, wherein said monoclonal antibody or antigen-binding fragment thereof comprises an HU177 monoclonal antibody or an antigen-binding fragment thereof.

20. The method of any one of the preceding claims, wherein administration of the antagonist of collagen or the functional fragment thereof increases CD8+ cells in a tumor in the subject by at least 1.1-fold, at least 1.2-fold, at least 1.5-fold, at least 2-fold, at least 3-fold, at least 4-fold, at least 5-fold, at least 10-fold, compared to a control.

21. The method of any one of the preceding claims, wherein administration of the antagonist of collagen or the functional fragment thereof increases CD8+ cells in ascites fluid in the subject by at least 1.1-fold, at least 1.2-fold, at least 1.5-fold, at least 2-fold, at least 3-fold, at least 4-fold, at least 5-fold, at least 10-fold, compared to a control.

22. The method of any one of the preceding claims, wherein administration of the antagonist of collagen or the functional fragment thereof decreases CD4+ Treg cells in a tumor in the subject by at least 1.1-fold, at least 1.2-fold, at least 1.5-fold, at least 2-fold, at least 3-fold, at least 4-fold, at least 5-fold, at least 10-fold, compared to a control.

23. The method of any one of the preceding claims, wherein administration of the antagonist of collagen or the functional fragment thereof decreases CD4+ Treg cells in ascites fluid in the subject by at least 1.1-fold, at least 1.2-fold, at least 1.5-fold, at least 2-fold, at least 3-fold, at least 4-fold, at least 5-fold, at least 10-fold, compared to a control.

24. The method of any one of the preceding claims, wherein administration of the antagonist of collagen or the antigen-binding fragment thereof decreases MDSC cells in a tumor in the subject by at least 1.1-fold, at least 1.2-fold, at least 1.5-fold, at least 2-fold, at least 3-fold, at least 4-fold, at least 5-fold, at least 10-fold, compared to a control.

25. The method of any one of the preceding claims, wherein the migration of T-cells to the tumor in the subject is increased compared to a control.

26. The method of any one of the preceding claims, wherein the migration of CD8+ T-cells to the tumor in the subject is increased compared to a control.

27. The method of any one of the preceding claims, wherein the migration of CD4+ Treg-cells and/or MDSCs to the tumor in the subject is decreased compared to a control.

28. The method of any one of the preceding claims, wherein the level of phosphorylation and/or activation of a P38 MAP Kinase in the subject is inhibited compared to a control.

29. The method of any one of claims 20-28. wherein the control is prior to administration of the adoptive cell therapeutic composition, and the antagonist of collagen or the antigen-binding fragment thereof.

30. The method of any one of the preceding claims, further comprising administering an antagonist of an integrin to the subject.

31. The method of claim 30, wherein the integrin comprises integrin αvβ3.

32. The method of claim 31, wherein said antagonist of integrin αvβ3 comprises an antibody capable of binding an RGDKGE (SEQ ID NO: 1) containing collagen epitope.

33. The method of any one of the preceding claims, wherein the adoptive cell therapeutic composition and the antagonist of collagen or the functional fragment thereof are administered simultaneously to the subject.

34. The method of any one of claims 1-32, wherein the adoptive cell therapeutic composition and the antagonist of collagen or the functional fragment thereof are administered consecutively, in any order, to the subject.

35. The method of any one of claims 30-32, wherein the adoptive cell therapeutic composition, the antagonist of collagen or the functional fragment thereof, and the antagonist of an integrin are administered simultaneously to the subject.

36. The method of any one of claims 30-32, wherein the adoptive cell therapeutic composition, the antagonist of collagen or the functional fragment thereof, and the antagonist of an integrin to the subject is conducted consecutively, in any order.

37. The method of any one of the preceding claims, wherein there is a time period of one minute to four weeks between the consecutive administration of the adoptive cell therapeutic composition and the antagonist of collagen or the functional fragment thereof and/or there are several administrations of the adoptive cell therapeutic composition and the antagonist of collagen or the functional fragment thereof.

38. The method of any one of the preceding claims, wherein the administration of the adoptive cell therapeutic composition and/or the antagonist of collagen or the functional fragment thereof is conducted through an intra-tumoral, intra-arterial, intravenous, intrapleural, intravesicular, intracavitary or peritoneal injection, or an oral administration.

39. The method of any one of the preceding claims, further comprising administering concurrent or sequential radiotherapy, monoclonal antibodies, chemotherapy, immunotherapy or other anticancer drugs or interventions to the subject.

40. The method of any one of the preceding claims, wherein the immunotherapy comprises administration of an immune checkpoint inhibitor to the subject.

41. The method of claim 40, wherein the immune checkpoint inhibitor comprises an inhibitor of CTLA-4, PD-1, PDL-1, Lag3, LAIR1, or LAIR 2.

42. The method of claim 40 or 41, wherein the immune checkpoint inhibitor comprises an anti-CTLA-4 antibody, an anti-PD-1 antibody, an anti-PDL-1 antibody, an anti-Lag3 antibody, an anti-LAIR1 antibody, or an anti-LAIR 2 antibody.

43. The method of any one of claims 40-42, wherein the immune checkpoint inhibitor comprises an anti-PD-1 antibody, or an anti-PDL-1 antibody.

44. The method of any one of the preceding claims, wherein the subject is a human.

45. The method of any one of the preceding claims, wherein the cancer is selected from the group comprising of melanoma, central nervous system (CNS) cancer, CNS germ cell tumor, lung cancer, leukemia, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, or hematological cancer.

46. The method of claim 45, wherein the hematological cancer is a leukemia or a lymphoma, optionally wherein the leukemia is acute lymphoblastic leukemia (ALL), acute myelogenous leukemia (AML), chronic lymphocytic leukemia (CLL), small lymphocytic lymphoma (SLL), chronic myelogenous leukemia (CML) or acute monocytic leukemia (AMoL).

47. The method of claim 45, wherein the lymphoma is follicular lymphoma, Hodgkin's lymphoma, or Non-Hodgkin's lymphoma, optionally wherein the Hodgkin's lymphoma is Nodular sclerosing subtype, mixed-cellularity subtype, lymphocyte-rich subtype, or lymphocyte depleted subtype.

48. The method of any one of claims 1-45, wherein the cancer is a solid cancer comprising melanoma, unresectable melanoma, metastatic melanoma, renal cancer, renal cell carcinoma, prostate cancer, metastatic castration resistant prostate cancer, ovarian cancer, epithelial ovarian cancer, metastatic epithelial ovarian cancer, breast cancer, triple negative breast cancer, lung cancer, and/or non-small cell lung cancer.

49. The method of any one of claims 1-45, wherein the cancer is melanoma.

50. The method of any one of claims 1-45, wherein the cancer is ovarian cancer.

51. A method of treating an inflammatory disease or disorder in a subject, the method comprising administering to the subject
a) an adoptive cell therapeutic composition; and
b) an antagonist of collagen or a functional fragment thereof,
thereby treating the autoimmune disease or disorder in the subject.

52. The method of claim 51, wherein the adoptive cell therapeutic composition comprises a cell type selected from a group consisting of a tumor infiltrating lymphocytes (TIL), T-cell receptor modified lymphocytes, and chimeric antigen receptor modified lymphocytes.

53. The method of claim 52, wherein the adoptive cell therapeutic composition comprises tumor infiltrating lymphocytes (TIL).

54. The method of any one of claims 51-53, wherein the adoptive cell therapeutic composition comprises a cell type selected from a group consisting of T-cells, CD8+ cells, CD4+ cells, NK-cells, delta-gamma T-cells, regulatory T-cells, and peripheral blood mononuclear cells.

55. The method of claim 54, wherein the adoptive cell therapeutic composition comprises T-cells.

56. The method of any one of claims 51-55, wherein the collagen comprises collagen type-I, collagen type II, collagen type III, or collagen type-IV.

57. The method of claim 56, wherein the antagonist of collagen and functional fragment thereof comprises an antagonist of collagen type-IV or a functional fragment thereof.

58. The method of claim 57, wherein the antagonist of collagen type-IV or the functional fragment thereof comprises an antagonist of the XL313 cryptic collagen epitope, or an antagonist of the HU177 cryptic collagen epitope.

59. The method of claim 58 wherein the antagonist of collagen type-IV or the functional fragment thereof comprises an antibody or an antigen-binding fragment thereof that binds a cryptic RGDKGE (SEQ ID NO: 1) containing collagen epitope.

60. The method of claim 58, wherein the antagonist of collagen type-IV or the functional fragment thereof comprises an antibody or an antigen-binding fragment thereof that binds a cryptic CPGFPGFC (SEQ ID NO: 16) containing collagen epitope.

61. The method of claim 59 or 60, wherein the antibody or the antigen-binding fragment thereof comprises a monoclonal antibody or an antigen-binding fragment thereof.

62. The method of claim 61, wherein said monoclonal antibody or antigen-binding fragment thereof comprises an XL313 monoclonal antibody or an antigen-binding fragment thereof.

63. The method of claim 61, wherein said monoclonal antibody or antigen-binding fragment thereof comprises an HU177 monoclonal antibody or an antigen-binding fragment thereof.

64. The method of any one of any one of claims 51-63, wherein the migration of T-cells to the inflammation in the subject is decreased.

65. The method of any one of claims 51-63, wherein the migration of CD8+ T-cells to inflammation in the subject is increased.

66. The method of any one of any one of claims 51-63, wherein the migration of CD4+ Treg-cells and/or MDSCs to inflammation in the subject is decreased.

67. The method of any one of claims 51-64, wherein the level of phosphorylation and/or activation of a P38 MAP Kinase in the subject is inhibited compared to a control.

68. The method of any one of claims 51-67, further comprising administering an antagonist of an integrin to the subject.

69. The method of claim 68, wherein the integrin comprises integrin αvβ3.

70. The method of claim 69, wherein said antagonist of integrin αvβ3 comprises an antibody capable of binding an RGDKGE (SEQ ID NO: 1) containing collagen epitope, or a CPGFPGFC (SEQ ID NO: 16) containing collagen epitope.

71. The method of any one of t claims 51-70, wherein the adoptive cell therapeutic composition and the antagonist of collagen or the functional fragment thereof are administered simultaneously to the subject.

72. The method of any one of claims 51-70, wherein the adoptive cell therapeutic composition and the antagonist of collagen or the functional fragment thereof are administered consecutively, in any order, to the subject.

73. The method of any one of claims 68-70, wherein the adoptive cell therapeutic composition, the antagonist of collagen or the functional fragment thereof, and the antagonist of an integrin are administered simultaneously to the subject.

74. The method of any one of claims 68-70, wherein the adoptive cell therapeutic composition, the antagonist of collagen or the functional fragment thereof, and the antagonist of an integrin to the subject is conducted consecutively, in any order.

75. The method of any one of claims 51-74, wherein there is a time period of one minute to four weeks between the consecutive administration of the adoptive cell therapeutic composition and the antagonist of collagen or the functional fragment thereof and/or there are several administrations of the adoptive cell therapeutic composition and the antagonist of collagen or the functional fragment thereof.

76. The method of any one of claims 51-75, wherein the administration of the adoptive cell therapeutic composition and/or the antagonist of collagen or the functional fragment thereof is conducted through an intra-tumoral, intra-arterial, intravenous, intrapleural, intravesicular, intracavitary or peritoneal injection, or an oral administration.

77. The method of any one of claims 51-76, wherein the inflammatory disease or disorder is selected from the group consisting of an allergy, ankylosing spondylitis, asthma, atopic dermatitis, an autoimmune disease or disorder, a cancer, celiac disease, chronic obstructive pulmonary disease (COPD), chronic peptic ulcer, cystic fibrosis, diabetes, glomerulonephritis, gout, hepatitis, an immune-mediated disease or disorder, inflammatory bowel disease (IBD), myositis, osteoarthritis, pelvic inflammatory disease (PID), multiple sclerosis, neurodegenerative diseases of aging, a periodontal disease, reperfusion injury transplant rejection, psoriasis, pulmonary fibrosis, rheumatic disease, scleroderma, sinusitis, dermatitis, pneumonitis, colitis and tuberculosis.

78. The method of any one of the claims 51-77, wherein the inflammatory disease or disorder is an autoimmune disease or disorder.

79. The method of claim 78, wherein the autoimmune disease or disorder is selected from the group consisting of Psoriasis, Graft-vs-Host Disease, Amyotrophic Lateral Sclerosis, Pemphigus Vulgaris, Systemic Lupus Erythematosus, Scleroderma, Ulcerative Colitis, Crohn's Disease, Type 1 Diabetes, Multiple Sclerosis, Alopecia Areata, Uveitis, Neuromyelitis Optica, Graves' disease, Hashimoto's thyroiditis, rheumatoid arthritis and Duchenne Muscular Dystrophy.

80. A pharmaceutical kit comprising an adoptive cell therapeutic composition and an antagonist of collagen or a functional fragment thereof, wherein the adoptive cell therapeutic composition is formulated in a first formulation and the antagonist of collagen or the functional fragment thereof are formulated in a second formulation.

81. The pharmaceutical kit of claim 80, further comprising an antagonist of an integrin, wherein the antagonist of an integrin is formulated in a third formulation.

82. The pharmaceutical kit of claim 80 or 81, wherein the first formulation and the second formulation and/or third formulation are for simultaneous or sequential, in any order, administration to a subject.


SEQUENCE LISTING (16 sequences)

SEQ ID NO: 1 - protein, 6 aa, synthetic construct
  REGION 1..6: note = Synthetic: collagen epitope
RGDKGE

SEQ ID NO: 2 - protein, 9 aa, synthetic construct
  REGION 1..9: note = Synthetic: P-1
KGDRGDAPG

SEQ ID NO: 3 - protein, 9 aa, synthetic construct
  REGION 1..9: note = Synthetic: P-2
QGPRGDKGE

SEQ ID NO: 4 - protein, 11 aa, synthetic construct
  REGION 1..11: note = Synthetic: control peptide CP
CQGPGGAAGGC

SEQ ID NO: 5 - protein, 11 aa, synthetic construct
  REGION 1..11: note = Synthetic: Synthetic collagen RGD containing peptide P-1
CKGDRGDAPGC

SEQ ID NO: 6 - protein, 11 aa, synthetic construct
  REGION 1..11: note = Synthetic: Synthetic collagen RGD containing peptide P-2
CQGPRGDKGEC

SEQ ID NO: 7 - protein, 11 aa, synthetic construct
  REGION 1..11: note = Synthetic: Synthetic collagen RGD containing peptide P-3
CAGSRGDGGPC

SEQ ID NO: 8 - protein, 10 aa, synthetic construct
  REGION 1..10: note = Synthetic: Synthetic collagen RGD containing peptide P-4
CQGIRGDKGE

SEQ ID NO: 9 - protein, 11 aa, synthetic construct
  REGION 1..11: note = Synthetic: Synthetic collagen RGD containing peptide P-5
CRGPRGDQGPC

SEQ ID NO: 10 - protein, 11 aa, synthetic construct
  REGION 1..11: note = Synthetic: peptide control P-C
CQGPSGAPGEC

SEQ ID NO: 11 - protein, 6 aa, synthetic construct
  REGION 1..6: note = Synthetic: collagen peptides
RGDAPG

SEQ ID NO: 12 - protein, 9 aa, synthetic construct
  REGION 1..9: note = Synthetic: P-3
AGSRGDGGP

SEQ ID NO: 13 - protein, 9 aa, synthetic construct
  REGION 1..9: note = Synthetic: P-4
QGIRGDKGE

SEQ ID NO: 14 - protein, 9 aa, synthetic construct
  REGION 1..9: note = Synthetic: P-5
RGPRGDQGP

SEQ ID NO: 15 - protein, 9 aa, synthetic construct
  REGION 1..9: note = Synthetic: P-C
QGPSGAPGE

SEQ ID NO: 16 - protein, 8 aa, synthetic construct
  REGION 1..8: note = Synthetic: collagen epitope
CPGFPGFC